(19) United States
(12) Patent Application Publication
(10) Pub. No.: US 2026/0199122 A1
(43) Pub. Date: Jul. 16, 2026

(54) APPARATUS AND METHODS FOR RECEIVING DISCHARGED URINE

(71) Applicant: PUREWICK CORPORATION, Covington, GA (US)

(72) Inventors: Ashley Marie Johannes, Atlanta, GA (US); Eric Alan Rehm, Lawrenceville, GA (US); Andrew L Meyer, Atlanta, GA (US); Raymond J. Newton, Bonsall, CA (US); Jason Glithero, McDonough, GA (US)

(21) Appl. No.: 19/443,729

(22) Filed: Jan. 8, 2026

Related U.S. Application Data
(63) Continuation of application No. 16/433,773, filed on Jun. 6, 2019, now Pat. No. 12,539,227.
Application No. 16/433,773 is a continuation-in-part of application No. PCT/US2018/015968, filed on Jan. 30, 2018.
(60) Provisional application No. 62/452,437, filed on Jan. 31, 2017.

(51) Int. Cl. A61F 5/453; A61F 5/44; A61F 5/443; A61F 5/445; A61F 13/15; A61M 25/00

(52) U.S. Cl. CPC A61F 5/453; A61F 5/4404; A61F 5/4405; A61F 5/443; A61F 5/445; A61F 13/15; A61F 5/4401; A61F 2013/15146; A61M 25/0017

(57) ABSTRACT

A system suitable for collecting and transporting urine away from the body of a person or animal may include a urine collecting assembly having a body, a sealing flange, and a reservoir within the body and partially defined by the sealing flange. The sealing flange can define an opening such that the interior of the body is accessible via the opening. A peripheral edge of the opening can be configured to seal around a shaft of a penis of a user disposed through the opening. The urine collecting assembly can also include an outlet in fluidic communication with the reservoir. The urine collecting assembly can be arranged such that a fluid can flow into the body from the urethral opening of the user's penis, collect in the reservoir, and flow out of the outlet.

### CROSS-REFERENCE TO RELATED APPLICATIONS

This application is a continuation of U.S. application Ser. No. 16/433,773 filed Jun. 6, 2019, which is a continuation-in-part application of International Application No. PCT/US2018/015968 filed on Jan. 30, 2018, which claims priority to U.S. Provisional Application No. 62/452,437 filed on Jan. 31, 2017, the disclosures of each of which is incorporated herein, in their entirety, by this reference.

### TECHNICAL FIELD

The present disclosure relates generally to systems, apparatus, and methods for collecting and transporting urine away from the body of a person or animal.

### BACKGROUND

The embodiments described herein relate generally to collecting and transporting urine away from the body of a person or animal. In various circumstances, a person or animal may have limited or impaired mobility such that typical urination processes are challenging or impossible. For example, a person may experience or have a disability that impairs mobility. A person may have restricted travel conditions such as those experienced by pilots, drivers, and workers in hazardous areas. Additionally, sometimes urine collection is needed for monitoring purposes or clinical testing.

Urinary catheters, such as a Foley catheter, can be used to address some of these circumstances, such as incontinence. Unfortunately, however, urinary catheters can be uncomfortable, painful, and can lead to complications, such as infections. Additionally, bed pans, which are receptacles used for the toileting of bedridden patients, such as those in a health care facility, are sometimes used. Bed pans, however, can be prone to discomfort, spills, and other hygiene issues.

Males who suffer the most severe consequences of urinary incontinence, such as discomfort, rashes, and sores are typically elderly and often bedbound. They also require continuous assistance to maintain hygiene. Characteristics often found in these patients: they typically lay on their back, the size of the penis often decreases with age, skin rolls containing fat tissue cause the penis to recede, often pointing upward while in a laying position, patients have difficulty reaching the penis and manipulating devices. A urine capture device should be designed with reference to these characteristics.

Available solutions are typically for use while standing up (such as cups and funnels), with a urine discharge port opposite to the distal end of the penis. Other designs such as condom-style catheters are difficult for patients to manipulate, too often they are dimensionally incompatible; and they do not stay on reliably.

Thus, there is a need for a device capable of collecting urine from a person or animal, particularly a male, comfortably and with minimal contamination of the user and/or the surrounding area.

### SUMMARY

In an embodiment, a urine collecting assembly is disclosed. The urine collecting assembly includes a body including an open proximal end and an at least partially closed distal end. The body includes a ring at or near the open proximal end. The ring includes an outer surface and an opposing inner surface defining an opening. The ring includes an attachment mechanism. The attachment mechanism includes a protrusion extending from the outer surface of the ring. The body also includes a sheath extending from or near the open proximal end to or near the at least partially closed distal end. The sheath includes at least one fluid impermeable layer. The sheath at least partially defines a reservoir. Further, the body includes a sump at the at least partially closed distal end of the body. The sump defines an outlet.

In an embodiment, a stabilization accessory configured to attach a urine collecting assembly to a user is disclosed. The stabilization accessory includes a base portion including a bottom surface that is configured to abut one or more regions about a penis of the user. The stabilization accessory also includes a raised portion extending upwardly from the base portion. The raised portion includes a top surface and an inner surface defining an opening therethrough. The opening is configured to receive the penis of the user. The raised portion includes an attachment mechanism extending inwardly from the inner surface.

In an embodiment, a urine collecting system is disclosed. The urine collecting system includes a urine collecting assembly including a body. The body includes an open proximal end and an at least partially closed distal end. The body also includes a ring at or near the open proximal end. The ring includes an outer surface and an opposing inner surface defining a first opening. The ring further includes a first attachment mechanism. The first attachment mechanism includes a protrusion extending from the outer surface of the ring. The body also includes a sheath extending from or near the open proximal end to or near the at least partially closed distal end. The sheath includes at least one fluid impermeable layer. The sheath at least partially defines a reservoir. The body further includes a sump at the at least partially closed distal end of the body. The sump defines an outlet. The urine collecting system also includes a stabilization accessory configured to attach the urine collecting assembly to a user. The stabilization accessory includes a base portion including a bottom surface that is configured to abut one or more regions about a penis of the user. The stabilization accessory also includes a raised portion extending upwardly from the base portion. The raised portion includes a top surface. The raised portion includes an inner surface defining a second opening therethrough. The second opening is configured to receive the penis of the user and the urine collecting assembly. The raised portion includes a second attachment mechanism extending inwardly from the inner surface.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1 is a schematic block diagram of a urine collecting system, according to an embodiment.

FIG. 2A is a perspective view of a urine collecting assembly, and FIG. 2B is a cross-sectional side view of the urine collecting assembly, according to an embodiment.

FIG. 3A is a perspective view of a urine collecting assembly showing an outlet tubing associated with an outlet extending through a portion of a body of the urine collecting assembly and from the top of the body, according to an embodiment.

FIG. 3B is a top view of the urine collecting assembly showing the arrangement of the outlet tubing and the outlet relative to the body, according to an embodiment.

FIG. 3C is a schematic cross-sectional view of a urine collecting assembly according to another embodiment.

FIGS. 4A and 4B are top and bottom perspective views of the urine collecting assembly, respectively, according to an embodiment.

FIG. 4C is a top view of the inner layer of the body and the sealing flange, according to an embodiment.

FIG. 4D is a schematic cross-sectional view taken along line 4D-4D of FIG. 4A

FIG. 4E is a schematic cross-sectional view taken along line 4E-4E of FIG. 4D.

FIGS. 5A and 5B are an isometric view and a schematic cross-sectional view, respectively, of a urine collecting assembly, according to an embodiment.

FIGS. 5C and 5D are an isometric view and a cross-sectional view, respectively, of a urine collecting assembly, according to an embodiment.

FIG. 6A is a schematic top view of a stabilization accessory, according to an embodiment.

FIGS. 6B-6D show a top view, a front cross-sectional view, and a side view of a urine collecting system that includes the stabilization accessory engaged with the urine collecting assembly, according to an embodiment.

FIG. 7A is a top view of a stabilization accessory with an oblong shape, according to an embodiment.

FIG. 7B is a side view of a urine collecting system that includes the stabilization accessory engaged with the urine collecting assembly, according to an embodiment.

FIG. 8A is an isometric view of a stabilization accessory, according to an embodiment.

FIGS. 8B and 8C is an isometric view and a schematic cross-sectional view, respectively, of a urine collecting system that includes the stabilization accessory engaged with an urine collecting assembly, according to an embodiment.

FIG. 8D is a schematic cross-sectional view of a urine collecting system that includes a stabilization accessory engaged with a urine collecting assembly, according to an embodiment.

FIG. 9A is an isometric view of a first piece of a stabilization accessory (shown assembled in FIG. 9C), according to an embodiment.

FIG. 9B is an isometric view of the second piece of the stabilization accessory (shown assembled in FIG. 9C), according to an embodiment.

FIG. 9C is a schematic cross-sectional view of the assembled stabilization accessory, according to an embodiment.

FIG. 10A is a schematic cross-sectional view of a system that includes the urine collecting assembly of FIGS. 5A-5B disposed in a stabilization accessory, according to an embodiment.

FIG. 10B is a schematic cross-sectional view of a system that include the urine collecting assembly of FIGS. 5A-5B disposed in a stabilization accessory, according to an embodiment.

FIGS. 11A and 11B are a top plan view and a cross-sectional view, respectively, of a stabilization accessory, according to an embodiment.

FIG. 11C is an isometric view of a second attachment mechanism of a urine collecting assembly that is configured to be used in conjunction with the stabilization accessory of FIGS. 11A and 11B, according to an embodiment.

FIG. 12 is a top plan view of a stabilization accessory, according to an embodiment.

FIG. 13A is a side plan view of a stabilization accessory, according to an embodiment.

FIG. 13B is a side plan view of a stabilization accessory, according to an embodiment.

FIG. 14 is a top plan view of a stabilization accessory, according to an embodiment.

FIG. 15 is an isometric view of packaging that includes a stabilization accessory that is configured to be adhesively attached to an individual and a pad, according to an embodiment

FIGS. 16A and 16B are a schematic view and a schematic cut-away view, respectively of a urine collecting system disposed on a user in a position for use, according to an embodiment.

FIG. 17 is a schematic illustration of a urine collecting system disposed on the body of a user, according to an embodiment.

FIG. 18 is a flowchart illustrating a method of using a urine collecting assembly to collect urine from a user, according to an embodiment.

### DETAILED DESCRIPTION

A urine collecting system is disclosed that is suitable for collecting and transporting urine away from the body of a person or animal, particularly a male. The disclosed urine collecting system includes a urine collecting assembly that may include a body and/or a sealing flange. The body can have an interior region bounded by a fluid impermeable side wall having an open proximal end and an at least partially closed distal end. The sealing flange can be coupled (e.g., permanently or reversibly coupled) to the side wall near the proximal end thereof. The sealing flange can have an opening therethrough with a peripheral edge of the opening configured to seal around the shaft of a penis of a user disposed therethrough. The urine collecting assembly can further include a fluid reservoir that is the interior region of the body and, therefore, the fluid reservoir is at least partially defined by at least a portion of the side wall. The reservoir can also be partially defined by the sealing flange. The urine collecting assembly also includes a fluid outlet in fluid communication with the reservoir that may be, for example, adjacent to the sealing flange. The urine collecting assembly can be configured to be disposed with a user's penis disposed through the opening such that the urethral opening of the penis is disposed within the reservoir (e.g., disposed within the interior region of the body) and the shaft of the penis is in sealing relationship with the peripheral edge of the opening such that the urine collecting assembly is configured to receive urine discharged from the urethral opening into the reservoir, and to have the received urine withdrawn from the reservoir via the outlet.

In some embodiments, a method may include disposing in operative relationship with the urethral opening of a male user, a urine collecting system. The urine collecting system can include at least one of a body, a sealing flange, a fluid reservoir, a fluid outlet, or a stabilization accessory. The body can have an interior region bounded by a fluid impermeable side wall having a proximal end and an at least partially closed distal end. The sealing flange can be coupled to the side wall near the proximal end thereof and can have an opening therethrough with a peripheral edge. The fluid reservoir can be within the interior region of the body and defined by at least a portion of the side wall and by the sealing flange. The fluid outlet can be in fluid communication with the reservoir and adjacent to the sealing flange. The operative relationship can include the user's penis being disposed through the opening in the sealing flange in sealing relationship with the peripheral edge of the opening and with the urethral opening of the penis disposed within the reservoir. The method can include allowing urine discharged from the urethral opening to be received in the reservoir and allowing the received urine to be withdrawn from the reservoir via the fluid outlet.

As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof.

The embodiments described herein can be formed or constructed of one or more biocompatible materials. Examples of suitable biocompatible materials include metals, ceramics, or polymers. Examples of suitable metals include pharmaceutical grade stainless steel, gold, titanium, nickel, iron, platinum, tin, chromium, copper, and/or alloys thereof. Examples of polymers include nylons, polyesters, polycarbonates, polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, polyethylene terephthalate (PET), polytetrafluoroethylene (PTFE), and/or blends and copolymers thereof.

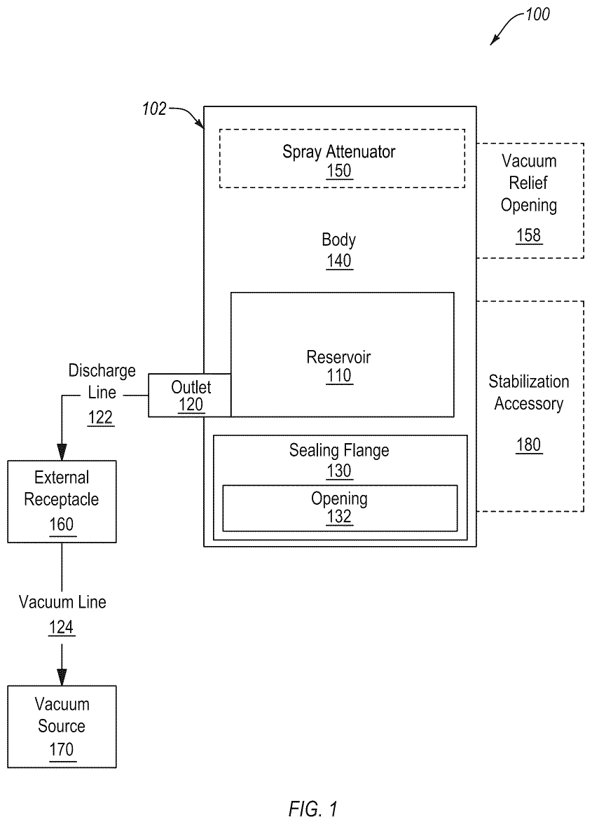

FIG. 1 is a schematic block diagram of a urine collecting system 100, according to an embodiment. The urine collecting system 100 includes a urine collecting assembly 102. The urine collecting assembly 102 can include at least one of a body 140, a sealing flange 130, a reservoir 110 within the body 140 that can be partially defined by the sealing flange 130, or a stabilization accessory 180. The sealing flange 130 can define an opening 132 such that the interior of the body 140 is accessible via the opening 132. A peripheral edge of the opening can be configured to seal around a shaft of a penis of a user disposed through the opening 132. The urine collecting assembly 102 also includes an outlet 120 in fluidic communication with the reservoir 110. The urine collecting assembly 102 can be arranged such that a fluid can flow into the body 140 from a urethral opening of the user's penis, collect in the reservoir 110, and flow out of the outlet 120. In an embodiment, the urine collecting assembly 102 can also include a spray attenuator 150 disposed within the body 140 and spaced from sealing flange 130, to attenuate spray from a stream of urine received into the body 140. The spray attenuator 150 can be, for example, a spun plastic material lining the interior portion of the top of body 140. In an embodiment, the urine collecting system 100 can include a discharge line 122. The discharge line 122 can be fluidly coupled to an external receptacle 160. The external receptacle 160 can be in fluidic communication with a vacuum source 170 via a vacuum line 124. The discharge line 122 and the vacuum line 124 can both include flexible tubing, such as, for example, flexible plastic tubing.

The reservoir 110 can be any suitable shape and/or size capable of collecting fluid received within reservoir 110. As described above, the reservoir 110 is defined by one or more fluid impermeable side walls of the body 140. In some embodiments the reservoir 110 can also be partially defined by the sealing flange 130 in combination with the one or more fluid impermeable side walls of the body 140. In an embodiment, the body 140 can be shaped as a cylindrical container. In some embodiments, the reservoir 110 is defined by one or more side walls of the body 140, the sealing flange 130, and an outer surface of a shaft of a penis of a user (not shown) disposed through the opening 132 defined by the sealing flange 130.

In an embodiment, the urine collecting assembly 102 can be sized such that the reservoir 110 is capable of collecting and temporarily holding a large or small amount of urine until the urine can be removed from the reservoir 110 via the outlet 120. For example, the urine collecting assembly 102 can be sized such that the reservoir 110 is configured to hold a small amount of urine as may be released due to incontinence. In an embodiment, the urine collecting assembly 102 can be sized such that the reservoir 110 is configured to hold a large amount of urine as may be released during voiding of a full bladder. In an embodiment, the urine collecting assembly 102 can be sized such that the reservoir 110 is configured to collect and hold a small or large amount of urine while the urine is simultaneously removed via, for example, gravity and/or a pump, such as the vacuum source 170. In a condition where the flow rate of urine into the urine collecting assembly 102 via the urethral opening of a user's penis is greater than the flow rate of urine through the discharge line 122, a temporary backup of urine may occur in the reservoir 110. Thus, the urine collecting assembly 102 can be sized such that the reservoir 110 can contain a volume of fluid that may temporarily accumulate due to the difference in flow rates into and out of the urine collecting assembly 102. Additionally, the urine collecting assembly 102 can be sized to accommodate anatomy of various shapes and sizes within the body 140 and via the opening 132.

Although the outlet 120 is shown as extending from the side of the reservoir 110, in an embodiment, the outlet 120 can extend from the bottom or top of the reservoir 110. For example, the outlet 120 can extend adjacent to or through a portion of the sealing flange 130. Positioning the outlet 120 lower in the reservoir 110 such that less or no urine can pool at the bottom of the reservoir 110 can allow for urine to be removed from the reservoir 110 more quickly and/or completely. In other embodiment, the outlet 120 can be positioned within the reservoir such that at least a portion of tubing associated with the outlet 120 extends from the top of the body 140. For example, a portion of tubing associated with the outlet 120 can extend from the reservoir 110 through at least a portion of the body 140. In such an embodiment, the outlet 120 can be positioned a distance from the reservoir 110 such that fluid can flow from the reservoir 110, through the tubing associated with the outlet 120, and from the outlet 120. In such an embodiment, positioning the reservoir end of the tubing associated with the outlet 120 towards the bottom of the reservoir 110 such that less or no urine can pool at the bottom of the reservoir 110 can allow for urine to be removed from the reservoir 110 more quickly and/or completely. In an embodiment, the outlet 120 can be disposed on the top of the body 140. Although the portion of tubing associated with the outlet 120 is described as extending through at least a portion of the body 140, in an embodiment the portion of tubing can be formed such that it is integral with a wall of the body 140. Said another way, a wall of the body 140 can define a lumen extending from the reservoir 110 to an outlet located above the reservoir 110, such as on the top of the body 140. The wall of the body 140 can define an inlet at the end of the lumen near the reservoir 110.

The external receptacle 160, via the discharge line 122, can collect fluid exiting the reservoir 110 through the outlet 120. The external receptacle 160 can be a sealed container. In an embodiment, the external receptacle 160 can be disposable. In an embodiment, the external receptacle 160 can be configured to be sterilized and reused.

In an embodiment, gravity can cause fluid within the reservoir 110 to follow a flow path (i.e., the fluid flow path including the outlet 120 and the discharge line 122) from the reservoir 110 to the external receptacle 160. In an embodiment, the vacuum source 170 can assist and/or provide the pressure differential needed to draw fluid voided from the urethral opening of a user into the body 140 into the reservoir 110, and from the reservoir 110 into the external receptacle 160. The vacuum source 170 can be fluidly coupled to the external receptacle 160 via a vacuum line 124 such that gaseous fluid is drawn from the external receptacle 160 via the vacuum line 124. As a result of the decrease in pressure within the external receptacle 160 caused by the drawing of gaseous fluid out of the external receptacle 160, liquid and/or gaseous fluid can be drawn from the reservoir 110, through the outlet 120, through the discharge line 122, and into the external receptacle 160. In an embodiment, the vacuum source 170 can apply sufficient suction to capture all or substantially all of the urine voided by a user in a variety of positions (e.g., when a user is lying on his side).

In an embodiment, the vacuum source 170 can be a pump that is readily available, inexpensive, relatively quiet, and/or configured to run continuously. For example, the vacuum source 170 can be a pump. The vacuum line 124 can be attached to the intake port of the pump (rather than the exhaust port) such that gaseous fluid is drawn into the pump from the external receptacle 160 via the vacuum line 124. In such an embodiment, the pump can have a configuration much like an aquarium aerator pump. In an embodiment, the necessary static vacuum of the urine collecting system 100 is about 3-10 feet of water (10%-30% of one atmosphere; 80-250 mm Hg) with a free-flow rate of about 10-100 cubic centimeters per second. In an embodiment, the necessary static vacuum of the urine collecting system 100 is higher or lower depending on the size of the user and the expected rate of urine flow from the user and/or through the urine collecting system 100. In an embodiment, the discharge line 122 can be about 0.25″ in diameter and the vacuum source 170 can be configured to cause about 500 cubic centimeters of urine to flow through the discharge line 122 to the external receptacle 160 over the duration of a typical urination event for a user, which may typically range from 10 to 20 seconds but may be shorter or longer, e.g., 5 to 90 seconds. In an embodiment, the vacuum source 170 can include a wall-mounted vacuum system, such as is found in hospitals. In an embodiment, a wall-mounted vacuum system can be configured to apply a vacuum of, for example, about 20 mm Hg to about 40 mm Hg. In an embodiment, the vacuum source 170 can be powered by electrical AC or DC power. For example, in mobile applications when the user is away from an AC power source, such as when the user is using the urine collecting system 100 during transportation via a wheel chair or motor vehicle, the vacuum source 170 can be powered by DC power. One suitable non-limiting example of a pump that can be used is the DryDoc Vacuum Station, available from PureWick, Inc.

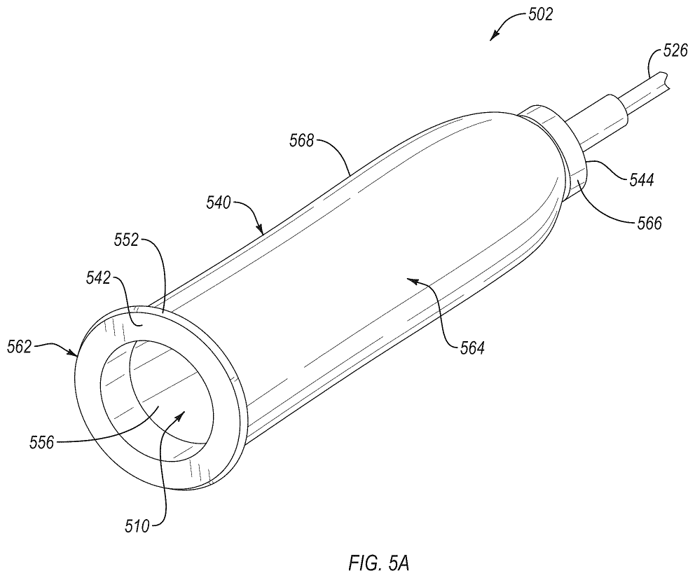

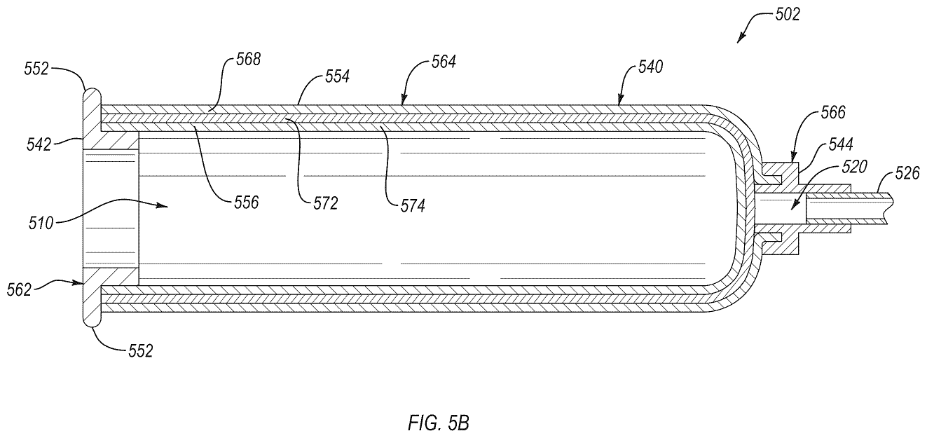

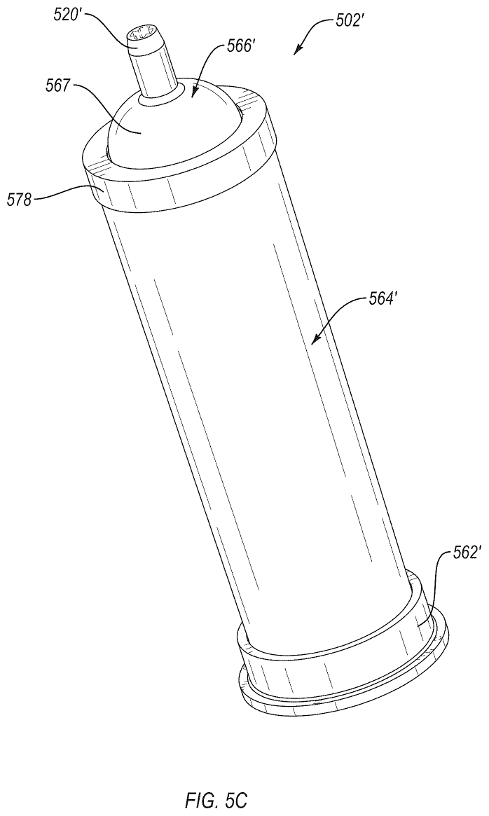

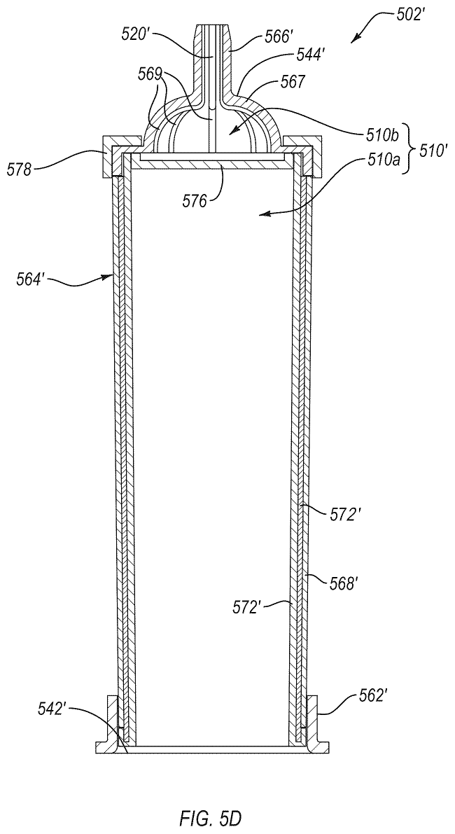

In an embodiment, the urine collecting system 100 can include a stabilization accessory 180 releasably coupleable to urine collecting assembly 102. The stabilization accessory 180 can be configured to receive the urine collecting assembly 102 within an opening (e.g., opening 582, 682, 782, 882, 982, 1082A, 1182, 1282, 1382A, 1382B, or 1482 of FIGS. 5A-14) defined by the stabilization accessory 180. The stabilization accessory 180 can be shaped and sized such that it can be disposed on a user's body. The stabilization accessory 180 can also be configured to maintain the urine collecting assembly 102 in a particular position and/or at a particular angle relative to the user's body via, for example, releasable frictional engagement between the urine collecting assembly 102 and the stabilization accessory 180. The stabilization accessory 180 can also stabilize the urine collecting assembly 102. In an embodiment, the stabilization accessory 180 and the urine collecting assembly 102 can be integrally formed with each other.

In an embodiment, the opening of the stabilization accessory 180 allows the urine collecting assembly 102 to rotate within the stabilization accessory 180 as a user of the urine collecting system 100 moves (e.g., rotates from side to side). In such an embodiment, the shape of the urine collecting assembly 102 and the opening of the stabilization accessory exhibit a circular cross-section (e.g., a generally cylindrical or conical shape) since other cross-sectional shapes, such as oblong shapes, can inhibit rotation of the urine collecting assembly 102 in the opening. Rotating the urine collecting assembly 102 within the opening of the stabilization accessory 180 can enable the outlet 120 to be oriented in the direction of the discharge line 122, thereby preventing kinks in the discharge line 122, prevent leaks forming between the user and the urine collecting assembly 102, etc., as the user moves. Additionally, if the urine collecting system 100 did not include the stabilization accessory 180, the body 140 of the assembly 102 may need to be adhesively attached to a region about the user's penis to prevent leaks between the body 140 and the region about the user's penis. However, adhesively attaching the body 140 to the region about the user's penis can cause the body 140 to pull and twist the region about the user's penis as the user moves.

The stabilization accessory 180 and/or the assembly 102 can be configured to enable the assembly 102 to rotate in the opening of the stabilization accessory 180 using any suitable method. In an embodiment, the opening of the stabilization accessory 180 can exhibit a size and shape that corresponds to, but is slightly larger than the urine collecting assembly 102 which can enable the urine collecting assembly 102 to rotate in the opening of the stabilization accessory. It is noted that any gap formed between the stabilization accessory 180 and the urine collecting assembly 102 is sufficiently small to substantially inhibit fluid flow therethrough. However, the gap between the stabilization accessory 180 and the urine collecting assembly 102 may be configured allow air to flow therein. In an embodiment, the stabilization accessory 180 and/or the urine collecting assembly 102 are configured to minimize friction therebetween which can facilitate rotation of the urine collecting assembly 102 in the opening of the stabilization accessory. For example, the stabilization accessory 180 and/or the urine collecting assembly 102 can at least one of be polished, include a low friction material, or include a lubricant that at least partially coats a surface thereof.

The stabilization accessory 180 can be any suitable shape and size, such as, for example, round, oblong, pie-shaped, or any other suitable shape, as shown in FIGS. 5A-14. In an embodiment, the stabilization accessory 180 can be shaped to conform to the shape of a user's body. In an embodiment, the stabilization accessory 180 can be configured to maintain the urine collecting assembly 102 at an angle that is about 90° relative to an axis running along the length of a user lying supine. In an embodiment, the stabilization accessory 180 can be configured to maintain the urine collecting assembly 102 at an angle that is greater than 90° or less than 90° (e.g., about 120°) relative to an axis running along the length of a user lying supine. In an embodiment, the stabilization accessory 180 can include an opening for the passage of a discharge line 122 from the urine collecting assembly 102. In an embodiment, the stabilization accessory 180 can be secured to the user's body via, for example, adhesive tape (e.g., via a hydrocolloid adhesive).

In an embodiment, the body 140 can define one or more vacuum relief openings 158 in fluid communication with the interior of body 140. The one or more vacuum relief openings 158 can allow gaseous fluid to flow into the body 140 from the external environment to prevent the development of a pressure differential within the urine collecting assembly 102 by the vacuum source 170 that is damaging or disruptive to the urine collecting assembly 102. Thus, the one or more vacuum relief openings 158 can prevent the body 140 from collapsing and can prevent the seal between the sealing flange 130 and a shaft of a penis disposed through the opening 132 from being broken due to a vacuum within the body 140. Said another way, the one or more vacuum relief openings 158 can be located such that at least one additional airflow path exists in the urine collecting assembly 102. The one or more vacuum relief openings 158 can be disposed at any suitable location on the body 140. In an embodiment, the one or more vacuum relief openings 158 can be disposed near the outlet 120 of the urine collecting assembly 102. In an embodiment, the one or more vacuum relief openings 158 can be disposed in a location that reduces the likelihood that the skin of the user inadvertently covers the hole, such as a location near the top of the body 140. In an embodiment, the one or more vacuum relief openings 158 can be disposed in a location that reduces the likelihood that liquid fluid (e.g., urine) will exit the urine collecting assembly 102 via the one or more vacuum relief openings 158. In an embodiment, the stabilization accessory 180 may define one or more vacuum relief openings.

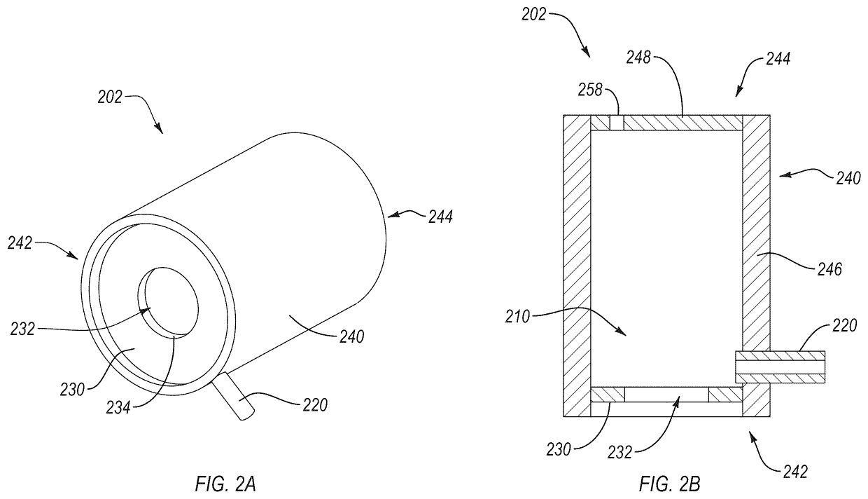

FIG. 2A is a perspective view of a urine collecting assembly 202, and FIG. 2B is a cross-sectional side view of the urine collecting assembly 202, according to an embodiment. The urine collecting assembly 202 can be the same or similar in structure and/or function as any of the urine collecting assemblies described herein, such as urine collecting assembly 102. For example, the urine collecting assembly 202 can include at least one of a body 240, a sealing flange 230, or a reservoir 210 (best shown in FIG. 2B) within the body 240 and partially defined by the sealing flange 230. The sealing flange 230 defines an opening 232 such that the interior of the body 240 is accessible via the opening 232. A peripheral edge 234 of the opening 232 is configured to seal around a shaft of a penis of a user disposed through the opening 232. The urine collecting assembly 202 also includes an outlet 220 in fluidic communication with the reservoir 210. The urine collecting assembly 202 can be arranged such that a fluid can flow into the body 240 (e.g., via a urethral opening of a user's penis disposed within the body 240), collect in the reservoir 210, and flow out of the outlet 220.

The body 240 has a fluid impermeable side wall 246 and a fluid impermeable end wall 248. The sealing flange 230 is coupled to the body 240 such that the body 240 in combination with the sealing flange 230 form a cylindrical container with an open proximal end 242 formed by the sealing flange 230 defining the opening 232 and an at least partially closed distal end 244 formed and closed by the end wall 248. The sealing flange 230 can be flexible and elastic such that the peripheral edge 234 of the sealing flange 230 can seal around an outer surface of a shaft of a penis of a user (not shown) disposed through the opening 232 defined by the sealing flange 230. For example, the sealing flange 230 can be formed from a polymer. Thus, the reservoir 210 can be defined by the sealing flange 230 in combination with the side wall 246 of the body 240, and an outer surface of a shaft of a penis of a user disposed through the opening 232.

The urine collecting assembly 202 can be sized such that the reservoir 210 is capable of collecting and temporarily holding a large or small amount of urine until the urine can be removed from the reservoir 210 via the outlet 220. For example, the urine collecting assembly 202 can be sized such that the reservoir 210 is configured to hold a small amount of urine as may be released due to incontinence. In an embodiment, the urine collecting assembly 202 can be sized such that the reservoir 210 is configured to hold a large amount of urine as may be released during voiding of a full bladder. In an embodiment, the urine collecting assembly 202 can be sized such that the reservoir 210 is configured to collect and hold a small or large amount of urine while the urine is simultaneously removed via, for example, gravity and/or a pump, such as a vacuum source the same or similar to the vacuum source 170. In a condition where the flow rate of urine into the urine collecting assembly 202 via the urethral opening of a user's penis is greater than the flow rate of urine through the outlet 220, a temporary backup of urine may occur in the reservoir 210. Thus, the urine collecting assembly 202 can be sized such that the reservoir 210 can contain a volume of fluid that may temporarily accumulate due to the difference in flow rates into and out of the urine collecting assembly 202. Additionally, the urine collecting assembly 202 can be sized to accommodate anatomy of various shapes and sizes within the body 240 and via the opening 232.

The outlet 220 extends from the side wall 246 of the body 240 (and thus from the side of the reservoir 210). An external receptacle (not shown) can be coupled to the outlet 220 via a discharge line (not shown) such that fluid (e.g., urine) exiting the reservoir 210 via the outlet 220 can be collected. The external receptacle and the discharge line can be the same or similar as the external receptacle 160 and the discharge line 122 described above. In an embodiment, gravity can cause fluid within the reservoir 210 to follow a flow path (i.e., the fluid flow path including the outlet 220 and the discharge line) from the reservoir 210 to the external receptacle. In an embodiment, a vacuum source (not shown), which can be the same or similar to vacuum source 170 described above, can assist and/or provide the pressure differential needed to draw fluid voided from the urethral opening of a user into the body 240 into the reservoir 210, and from the reservoir 210 into the external receptacle. In an embodiment, the vacuum source can apply sufficient suction to capture all or substantially all of the urine voided by a user that is collected at the bottom of the urine collecting assembly 202 (i.e., the open proximal end 242) near the outlet 220.

In an embodiment (not shown), the urine collecting assembly 202 can also include a spray attenuator disposed within the body 240 and spaced from sealing flange 230, to attenuate spray from a stream of urine received into the body 240. In an embodiment, the body 240 can define at least one vacuum relief opening 258.

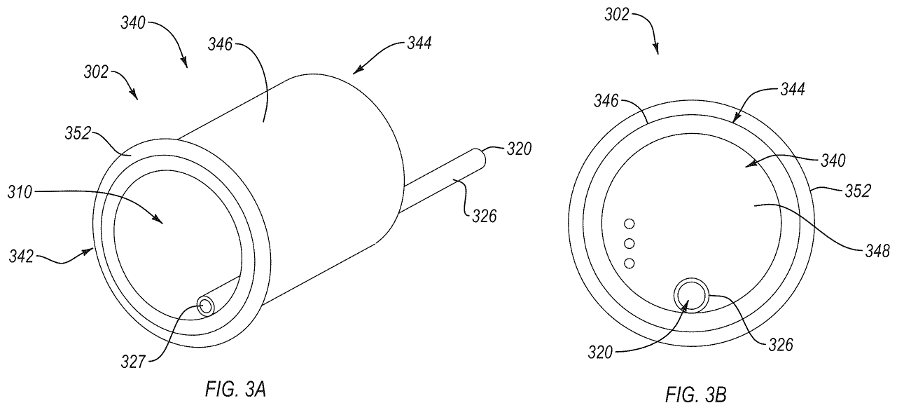

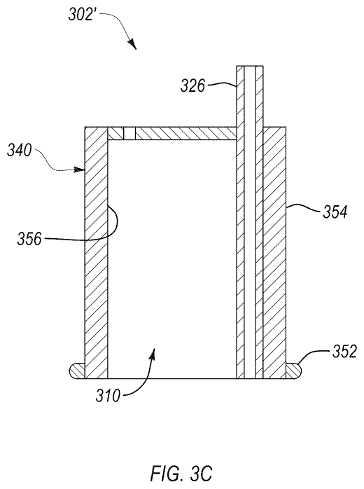

Although the outlet 220 of the urine collecting assembly 202 is shown as extending from the side wall 246 of the urine collecting assembly 202, in some embodiments the outlet can extend from the top of the urine collecting assembly. For example, FIG. 3A is a perspective view of a urine collecting assembly 302 showing an outlet tubing 326 associated with an outlet 320 extending through a portion of a body 340 of the urine collecting assembly 302 and from the top of the body 340, according to an embodiment. FIG. 3B is a top view of the urine collecting assembly 302 showing the arrangement of the outlet tubing 326 and the outlet 320 relative to the body 340, according to an embodiment. Extending the outlet 320 through the top of the body 340 can facilitate usage of the urine collecting assembly 302 with a stabilization accessory since the stabilization accessory would not need to define an opening for the discharge line to pass through.

The urine collecting assembly 302 can be the same or similar in structure and/or function as any of the urine collecting assemblies described herein, such as urine collecting assemblies 102, 202. For example, the urine collecting assembly 302 includes a reservoir 310 within the body 340. However, the urine collecting assembly 302, as illustrated, does not include a flange though, in some embodiments, the urine collecting assembly 302 can include a flange that is similar to the sealing flange 230 shown in FIGS. 2A and 2B. Omitting the sealing flange from the urine collecting assembly 302 allows the urine collecting assembly 302 to be used, in conjunction with a stabilization accessory (not shown), with a penis that exhibits a diameter or a length that is too small to be sealed with a sealing flange. As described above, the urine collecting assembly 302 also includes the outlet 320 in fluid communication with the reservoir 310 via the outlet tubing 326. The urine collecting assembly 302 can be arranged such that a fluid can flow into the body 340 (e.g., via a urethral opening of a user's penis disposed within the body 340), collect in the reservoir 310, and flow into the outlet tubing 326 via an inlet 327, through the outlet tubing 326, and out of the outlet 320.

The body 340 has a fluid impermeable side wall 346 and a fluid impermeable end wall 348. The body 340, optionally in combination with a sealing flange, can form a cylindrical container with an open proximal end 342 and an at least partially closed distal end 344 formed and closed by the end wall 348. Thus, the reservoir 310 can be defined by at least one of the sealing flange, the side wall 346 of the body 340, and an outer surface of a shaft of a penis of a user disposed through the opening 232.

The urine collecting assembly 302 can be sized such that the reservoir 310 is capable of collecting and temporarily holding a large or small amount of urine until the urine can be removed from the reservoir 310 via the outlet 320. For example, the urine collecting assembly 302 can be sized such that the reservoir 310 is configured to hold a small amount of urine as may be released due to incontinence. In an embodiment, the urine collecting assembly 302 can be sized such that the reservoir 310 is configured to hold a large amount of urine as may be released during voiding of a full bladder. In an embodiment, the urine collecting assembly 302 can be sized such that the reservoir 310 is configured to collect and hold a small or large amount of urine while the urine is simultaneously removed via, for example, gravity and/or a pump, such as a vacuum source the same or similar to the vacuum source 170. In a condition where the flow rate of urine into the urine collecting assembly 302 via the urethral opening of a user's penis is greater than the flow rate of urine through the outlet 320, a temporary backup of urine may occur in the reservoir 310. Thus, the urine collecting assembly 302 can be sized such that the reservoir 310 can contain a volume of fluid that may temporarily accumulate due to the difference in flow rates into and out of the urine collecting assembly 302. Additionally, the urine collecting assembly 302 can be sized to accommodate anatomy of various shapes and sizes within the body 340 and via the opening 332.

As described above, the outlet tubing 326 extends through a portion of the body 340. In an embodiment, the outlet tubing 326 can extend along an inner surface of the side wall 346 of the body 340. As shown in FIG. 3B, the outlet tubing 326 can extend through the end wall 348 and out of the top of the urine collecting assembly 302 such that the outlet 320 is disposed a distance from the top of the body 340. Thus, fluid can flow from the reservoir 310, through the outlet tubing 326, and from the outlet 120. In such an embodiment, positioning the inlet 327 of the outlet tubing 326 towards the bottom of the reservoir 310 such that less or no urine can pool at the bottom of the reservoir 310 can allow for urine to be removed from the reservoir 310 more quickly and/or completely.

An external receptacle (not shown) can be coupled to the outlet 320 via a discharge line (not shown) such that fluid (e.g., urine) exiting the reservoir 310 via the outlet tubing 326 and the outlet 320 can be collected. The external receptacle and the discharge line can be the same or similar as the external receptacle 160 and the discharge line 122 described above. In an embodiment, a vacuum source (not shown), which can be the same or similar to vacuum source 170 described above, can assist and/or provide the pressure differential needed to draw fluid voided from the urethral opening of a user into the body 340 into the reservoir 310, into the inlet 327, through the outlet tubing 326, and from the outlet 320 towards and/or into the external receptacle. In an embodiment, the vacuum source can apply sufficient suction to capture all or substantially all of the urine voided by a user that is collected at the bottom of the urine collecting assembly 302 (i.e., the open proximal end 342) near the inlet 327.

FIG. 3C is a schematic cross-sectional view of a urine collecting assembly 302′ according to another embodiment. Except as otherwise disclosed herein, the urine collecting assembly 302′ can be the same as or substantially similar to the urine collecting assembly 302 of FIGS. 3A-3B. For example, the urine collecting assembly 302′ can include a body 340, a reservoir 310 at least partially defined by the body 340, and a tubing 326.

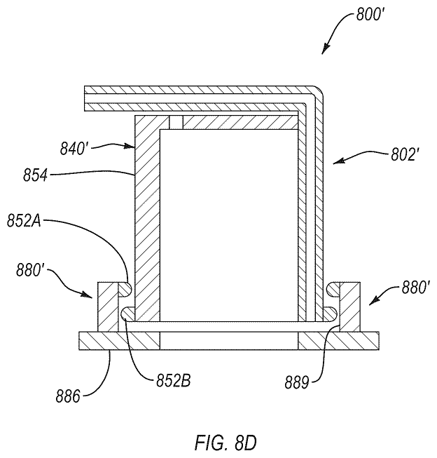

The urine collecting assembly 302′ includes at least one attachment mechanism 352 that is configured to reversibly couple the urine collecting assembly 302′ to a stabilization accessory (e.g., any of the stabilization accessories disclosed herein). In an embodiment, as illustrated, the attachment mechanism 352 can include at least one protrusion that extends from a surface of the body 340. The at least one protrusion can include a single protrusion (e.g., a nub), a plurality of protrusions (e.g., a plurality of nubs), a continuous annular protrusion extending around an entire circumference of the body 340, or any other suitable protrusion. The protrusion can extend from an external surface 354 of the body 340 (as shown) or can be configured to extend from an internal surface 356 of the body 340. The protrusion can be configured to interact with a feature of the stabilization accessory. For example, the protrusion can be configured to interact with a protrusion formed on a surface of the stabilization accessory (e.g., as illustrated in FIG. 8D) or be configured to be at least partially disposed in a recess formed in the stabilization accessory. As such, the protrusion can reversibly couple the urine collecting assembly 302′ to the stabilization accessory by sliding the protrusion of the urine collecting assembly 302′ over the protrusion of the stabilization accessory or sliding the protrusion of the urine collecting assembly 302′ into the recess of the stabilization accessory. One benefit of the illustrated attachment mechanism 352 is that the protrusion can enable the urine collecting assembly 302′ to rotate relative to the stabilization accessory. Further, the protrusion can prevent the urine collecting assembly 302′ from being decoupled from the stabilization accessory unless the urine collecting assembly 302′ is pulled from the stabilization accessory.

It is noted that the attachment mechanism 352 can include other elements instead of or in conjunction with the protrusion shown in FIG. 3C. For example, the attachment mechanism 352 can include threads that are configured to threadedly couple the urine collecting assembly 302′ to the stabilization accessory. In another example, the attachment mechanism 352 can include a convexly or concavely curved surface is configured to interface with a corresponding concavely or convexly curved surface of the stabilization accessory. In another example, the attachment mechanism 352 can include a recess formed therein that is configured to at least partially receive at least one protrusion extending from a surface of the stabilization accessory. In another example, the attachment mechanism 352 can include a magnet or a magnetically attractable material that is configured to interact with a magnet or magnetically attractable material of the stabilization accessory.

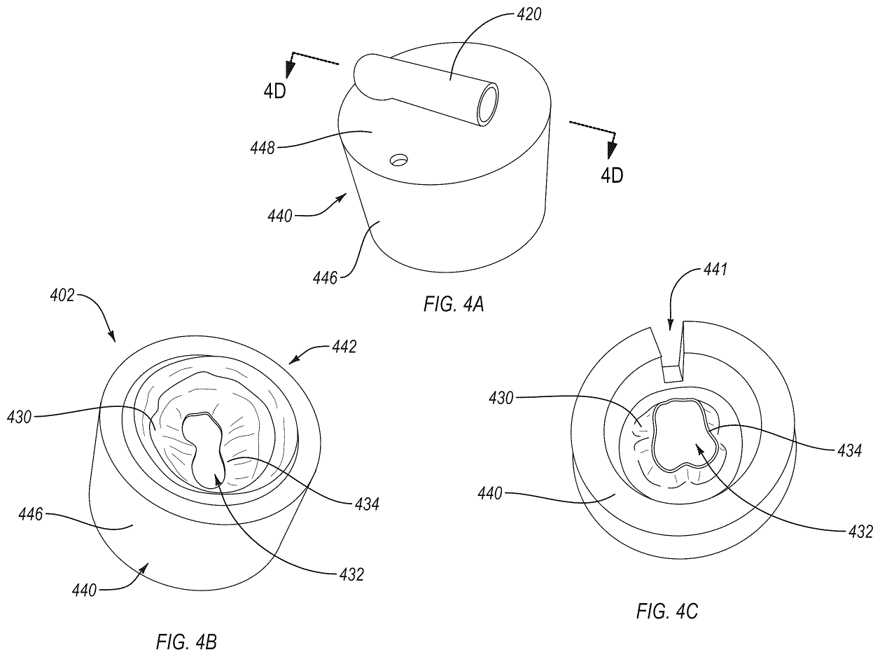

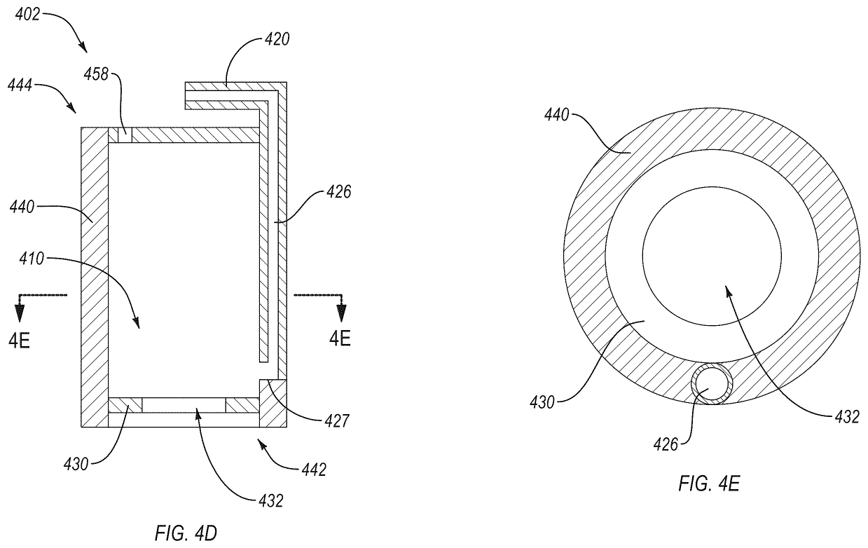

FIGS. 4A-4E illustrate a urine collecting assembly 402 according to another embodiment. FIGS. 4A and 4B are top and bottom perspective views of the urine collecting assembly 402, respectively, according to an embodiment. The urine collecting assembly 402 can be the same or similar in structure and/or function to any of the urine collecting assemblies described herein, such as the urine collecting assemblies 102, 202, or 302. For example, the urine collecting assembly 402 can include at least one of a body 440, a sealing flange 430, or a reservoir 410 (best shown in FIG. 4D, which is a schematic cross-sectional view taken along line 4D-4D of FIG. 4A) within the body 440 and partially defined by the sealing flange 430. The sealing flange 430 defines an opening 432 such that the interior of the body 440 is accessible via the opening 432. A peripheral edge 434 of the opening 432 is configured to seal around a shaft of a penis of a user disposed through the opening 432. The urine collecting assembly 402 also includes an outlet 420 in fluidic communication with the reservoir 410 via outlet tubing 426 (shown in FIG. 4D). The urine collecting assembly 402 can be arranged such that a fluid can flow into the body 440 (e.g., via a urethral opening of a user's penis disposed within the body 440), collect in the reservoir 410, and flow into the outlet tubing 426 via an inlet 427, through the outlet tubing 426, and out of the outlet 420.

The body 440 has a fluid impermeable side wall 446 and a fluid impermeable end wall 448 defining one or more vacuum relief openings 458. The sealing flange 430 can be coupled to the body 440 such that the body 440 in combination with the sealing flange 430 form a cylindrical container with an open proximal end 442 formed by the sealing flange 430 defining the opening 432 to the interior of the body 440 and an at least partially closed distal end 444 formed and closed by the end wall 448. The sealing flange 430 can be flexible and elastic such that the peripheral edge 434 of the sealing flange 430 can seal around an outer surface of a shaft of a penis of a user (not shown) disposed through the opening 432 defined by the sealing flange 430. For example, the sealing flange 430 can be formed from a polymer. Thus, the reservoir 410 can be defined by the sealing flange 430, the side wall 446 of the body 440, and an outer surface of a shaft of a penis of a user disposed through the opening 432. Although sealing flange 430 is shown in FIG. 4D as being recessed from the proximal end of body 440, in other embodiments the sealing flange can be flush with the proximal end of the body.

The urine collecting assembly 402 can be sized such that the reservoir 410 is capable of collecting and temporarily holding a large or small amount of urine until the urine can be removed from the reservoir 410 via the outlet 420. For example, the urine collecting assembly 402 can be sized such that the reservoir 410 is configured to hold a small amount of urine as may be released due to incontinence. In an embodiment, the urine collecting assembly 402 can be sized such that the reservoir 410 is configured to hold a large amount of urine as may be released during voiding of a full bladder. In an embodiment, the urine collecting assembly 402 can be sized such that the reservoir 410 is configured to collect and hold a small or large amount of urine while the urine is simultaneously removed via, for example, gravity and/or a pump, such as a vacuum source the same or similar to the vacuum source 170. In a condition where the flow rate of urine into the urine collecting assembly 402 via the urethral opening of a user's penis is greater than the flow rate of urine through the outlet 420, a temporary backup of urine may occur in the reservoir 410. Thus, the urine collecting assembly 402 can be sized such that the reservoir 410 can contain a volume of fluid that may temporarily accumulate due to the difference in flow rates into and out of the urine collecting assembly 402. Additionally, the urine collecting assembly 402 can be sized to accommodate anatomy of various shapes and sizes within the body 440 and via the opening 432.

As described above, the outlet tubing 426 extends through a portion of the body 440. The side wall 446 of the body 440 includes an inner layer and an outer layer. The inner layer can include, for example, a rectangle-shaped piece of spun plastic, or open or closed cell foam. The outer layer can include, for example, adhesive tape applied to the inner layer when the inner layer has been rolled or folded from a rectangular shape to maintain a cylindrical shape. The outer layer can be any other fluid impermeable material, if the inner layer is fluid permeable. Alternatively, if the inner layer is fluid impermeable, the outer layer may be omitted.

FIG. 4C is a top view of the inner layer of the body 440 and the sealing flange 430, according to an embodiment. The inner layer of the body 440 can define an elongated opening 441. The elongated opening 441 can be shaped and sized to receive the outlet tubing 426 (as shown in FIG. 4E, which is a schematic cross-sectional view taken along line 4E-4E of FIG. 4D) such that the outlet tubing 426 extends through the body 440, through the end wall 448, and out of the top of the urine collecting assembly 402. Thus, fluid can flow from the reservoir 410, through the outlet tubing 426, and from the outlet 420. In such an embodiment, positioning the inlet 427 of the outlet tubing 426 towards the bottom of the reservoir 410 such that less or no urine can pool at the bottom of the reservoir 410 can allow for urine to be removed from the reservoir 410 more quickly and/or completely.

An external receptacle (not shown) can be coupled to the outlet 420 via a discharge line (not shown) such that fluid (e.g., urine) exiting the reservoir 410 via the outlet tubing 426 and the outlet 420 can be collected. The external receptacle and the discharge line can be the same or similar as the external receptacle 160 and the discharge line 122 described above. In an embodiment, a vacuum source (not shown), which can be the same or similar to vacuum source 170 described above, can assist and/or provide the pressure differential needed to draw fluid voided from the urethral opening of a user into the body 440 into the reservoir 410, into the inlet 427, through the outlet tubing 426, and from the outlet 420 towards and/or into the external receptacle. In an embodiment, the vacuum source can apply sufficient suction to capture all or substantially all of the urine voided by a user that is collected at the bottom of the urine collecting assembly 402 (i.e., the open proximal end 442) near the inlet 427.

FIGS. 5A and 5B are an isometric view and a schematic cross-sectional view, respectively, of a urine collecting assembly 502, according to an embodiment. Except as otherwise disclosed herein, the urine collecting assembly 502 can be the same as or similar to any of the urine collecting assemblies disclosed herein. For example, the urine collecting assembly 502 can include a body 540 having an open proximal end 542 and an at least partially closed distal end 544, a reservoir 510 at least partially defined by the body 540, and tubing 526 fluidly coupled to the reservoir 510.

The body 540 of the urine collecting assembly 502 can include a ring 562 at or near the open proximal end 542 of the body 540, a sheath 564 extend from or near the open proximal end 542 to or near the at least partially closed distal end 544 of the body 540, and a sump 566 at the at least partially closed distal end 544 of the body 540. In an embodiment, the ring 562, the sheath 564, and the sump 566 are all distinct components from each other. In an embodiment, at least two of the ring 563, the sheath 564, or the sump 566 are integrally formed together (e.g., are formed from a single piece).

The sheath 564 is configured to prevent a fluid (e.g., urine) escaping from the reservoir 510 and to move the fluid towards the sump 566 and the tubing 526. As such, referring to FIG. 5B, the sheath 564 can include a plurality of layers that facilitate the operation of the sheath 564. For example, the sheath 564 can include a fluid impermeable layer 568, a fluid permeable layer 574 (e.g., a one-way fluid movement fabric, gauze, or cloth), and a fluid permeable support 572 positioned between the fluid permeable layer 574 and the fluid impermeable layer 568. The fluid permeable support 572 may include a porous layer (e.g., a spun polymer layer. The fluid impermeable layer 568 can form an external surface 554 of the body 540 and prevent the fluid from leaking through the sheath 564. The fluid permeable layer 574 can form an internal surface 556 of the body 540. The fluid permeable layer 574 can be configured to move the fluid from the reservoir 510 to the fluid permeable support 572 and may substantially prevent the fluid that is in the fluid permeable support 572 from flowing back into the reservoir 510. As such, the fluid permeable layer 574 can remove fluid from around a penis thereby leaving the penis dry. The fluid permeable support 572 can form an inner layer between the fluid permeable layer 574 and the fluid impermeable layer 568. The fluid permeable support 572 can enable the fluid to flow generally towards the tubing 526.

It is noted that one or more layers of the sheath 564 can be omitted. For example, the fluid permeable layer 574 can be omitted such that the fluid permeable support 572 forms the internal surface 556 of the body 540. In such an example, the sheath 564 can rely on the wicking ability of the fluid permeable support 572 and a suction force applied to the urine collecting assembly 502 to remove the fluid from the penis. In another example, the sheath 564 only include the fluid impermeable layer 568. In such an example, the sheath 564 can rely on the suction force applied to the urine collecting assembly 502 to remove the fluid from the penis. In another example, the sheath 564 only includes the fluid impermeable layer 568 and the fluid permeable layer 574. In such an example, the sheath 564 can form a channel (not shown) between the fluid impermeable layer 568 and the fluid permeable layer 574 and the channel is fluidly coupled to the tubing 526.

The fluid permeable layer 574 and/or the fluid permeable support 572 may include permeable material designed to wick or pass fluid therethrough. The permeable properties referred to herein may be wicking, capillary action, diffusion, or other similar properties or processes, and are referred to herein as “permeable” and/or “wicking.” Such “wicking” may not include absorption of fluid into the wicking material. Put another way, substantially no absorption of fluid into the material may take place after the material is exposed to the fluid and removed from the fluid for a time. While no absorption is desired, the term “substantially no absorption” may allow for nominal amounts of absorption of fluid into the wicking material (e.g., absorbency), such as less than about 10 wt % of the dry weight of the wicking material, less than about 7 wt %, less than about 5 wt %, less than about 3 wt %, less than about 2 wt %, less than about 1 wt %, or less than about 0.5 wt % of the dry weight of the wicking material. Wicking material can include natural fibers. In such examples, the material may have a coating to prevent or limit absorption of fluid into the material, such as a water repellent coating.

The sheath 564 is configured to have a penis disposed therein. To facilitate fluid collection and improve comfort, the sheath 564 can be flexible thereby allowing the sheath 564 to correspond to the shape of a penis. For example, the flexible sheath 564 can at least partially collapse when the penis is not erect and at least partially expand and bend to the shape of the penis as the penis becomes erect. Forming the layers of the sheath 564 from at least one of thin layers (e.g., less than 500 μm thick, and more particularly less than 250 μm thick, less than 100 μm thick, or less than 50 μm thick), flexible layers, or fabric can allow the sheath 564 to be sufficiently flexible.

The ring 562 can be more rigid than the sheath 564. For example, the ring 562 can be formed from a flexible polymer that is at least one of thicker than the entire sheath 564 or exhibits a Young's modulus that is greater than sheath 564. As such, the ring 562 can provide some structure at or near the open proximal end 542 of the body 540. The increased rigidity of the ring 562 can cause the open proximal end 542 to remain open thereby facilitating insertion of a penis into the urine collecting assembly 502. Further, in an embodiment, the increased rigidity of the ring 562 can enable the ring 562 to act as an attachment mechanism (e.g., similar to the attachment mechanism 352 of FIG. 3C). For example, as illustrated, the ring 562 can include at least one protrusion 552 that extends from the rest of the body 540. In another example, the ring 562 can define a recess, include threads, or include any other attachment mechanism disclosed herein.

The sump 566 is configured to attach the rest of the urine collecting assembly 502 to the tube 526. For example, the sump 566 can define an outlet 520 extending through at least the fluid impermeable layer 568 thereby coupling the tubing 526 to the fluid permeable support 572 and/or the reservoir 510. Further, the sump 566 can close the at least partially closed distal end 544 of the body 540. For example, the sump 566 can bunch up the sheath 564 and close any gaps that may form.

The ring 562, the sheath 564, the sump 566, and the tubing 526 can be attached together using any suitable method. For example, at least two of the ring 562, the sheath 564, the sump 566, or the tubing 526 can be attached together using at least one of an interference fit, an adhesive, stitching, welding (e.g., ultrasonic welding), tape, any other suitable method, or combinations thereof.

FIGS. 5C and 5D are an isometric view and a cross-sectional view, respectively, of a urine collecting assembly 502′, according to an embodiment. Except as otherwise disclosed herein, the urine collecting assembly 502′ may be the same as or substantially similar to any of the urine collecting assemblies disclosed herein. For example, similar to the urine collecting assembly 502 of FIGS. 5A and 5B, the urine collecting assembly 502′ may include a ring 562′, a sheath 564′ (e.g., at least one of a fluid impermeable layer 568′, a fluid permeable support 572′, or a fluid permeable layer 574′), and a sump 566′.

The urine collecting assembly 502′includes a reservoir 510′ that is configured to receive and at least temporarily store fluids from a user of the urine collecting system 500′ therein. The reservoir 510′ may include one or more sections. For example, the reservoir 510′ may include a first section 510a that is defined by the sheath 564′. However, as illustrated, the reservoir 510′ may include one or more additional sections that are defined by one or more additional components of the urine collecting assembly 502′. For example, sump 566′ may exhibit a bulbous portion 567 extending from an outlet 520′ of the sump 566′ towards an open proximal end 542′ of the sheath 564′. The bulbous portion 567 may define a second section 510b of the reservoir 510′. The second section 510b may provide a location to store the fluid that is spaced from a penis of a user that is partially disposed in the urine collecting assembly 502′. As such, the second section 510b may decrease the amount of fluids that contact the penis.

The sheath 564′ extends from or near an open proximal end 542′ of the urine collecting assembly 502′ to or near an at least partially closed distal end 544′ of the urine collecting assembly 502′. However, in an embodiment, the portion of the sheath 564′ at or near the at least partially closed distal end 544′ defines a hole (e.g., opening). In such an embodiment, the urine collecting assembly 502′ may include a porous top layer 576 that at least partially covers the hole. The porous top layer 576 may separate the first section 510a of the reservoir 510′ from the second section 510b of the reservoir 510′.

The porous top layer 576 may be the same as or substantially similar to any of the fluid impermeable supports or fluid permeable layers disclosed herein. For example, the porous top layer 576 may include a one-way fluid movement fabric, thereby allowing fluid to flow from the first section 510a of the reservoir 510′ to the second section 510b of the reservoir 510′ while preventing the fluid from flowing from the second section 510b back to the first section 510a. As such, the porous top layer 576 may decrease the amount of fluid that contacts the penis when the porous top layer 576 includes the one-way fluid movement fabric. However, it is noted that the porous top layer 576 may include gauze, other types of fabric, or any other suitable porous material instead of or in addition to the one-way fluid movement fabric.

As previously discussed, the sump 566′ includes a bulbous portion 567. The sump 566′ may also include one or more ridges 569 extending from or near the outlet 520′ towards the open proximal end 542′. In an embodiment, the ridges 569 may direct any fluids that are in the second section 510b of the reservoir 510′ towards the outlet 520. In an embodiment, the ridges 569 may provide at least some structural support to the bulbous portion 567 such that ridges 569 resist the deformation and/or collapse of the bulbous portion 567. It is noted that the bulbous portion 567 may include one or more structural elements instead of or in addition to the ridges 569. For example, the bulbous portion 567 may define one or more recesses that resist bending stresses.

The sump 566′ may be exposed to the atmosphere, which allows the sump 566′ to be visible, and may be exposed to an interior of the urine collecting assembly 502′. As such, the sump 566′ may provide fluid communication between the atmosphere and the interior of the urine collecting assembly 502′ and/or provide information. For example, the sump 566′ may be formed from a porous material that is configured to assist in suction balance. In an embodiment, the sump 566′ may be formed from a material that is configured to change color or state when exposed to bacteria to indicate that an individual using the urine collecting assembly 502′ has an infection.

The urine collecting assembly 502′ may include an upper ring 578. The upper ring 578 may be configured to be positioned adjacent to the portion of the sump 566′ that is connected to the sheath 564′. In an embodiment, the upper ring 578 provides additional strength and/or rigidity to the portion of the sump 566′ that is connected to the sheath 564′. For example, the sheath 564′ may be flexible, thereby allowing the sheath 564′ to conform to the environment as well as gravity to create a shape that conforms to the anatomy of the individual wearing the urine collecting assembly 502′ thus giving the urine collecting assembly 502′ an adjustable length. Meanwhile, the upper ring 578 provides some rigidity to the urine collecting assembly 502′, thereby inhibiting leakage and patient injuries that the sheath 502′, by itself, cannot prevent. In an embodiment, the upper ring 578 may be connected to the sheath 564′. In such an embodiment, the upper ring 578 may be connected to the sheath 564′ along with the sump 566′ thereby increasing the attachment strength between the sheath 564′ and the sump 566′ or the upper ring 578 may indirectly connect the sump 566′ to the sheath 564′ (e.g., only the upper ring 578 is directly connected to the sheath 564′).

In an embodiment, the upper ring 578 may include a writeable area that is configured to receiving one or more markings thereon, for example, from a pencil or marker. The writeable area of the upper ring 578 may allow medical practitioners to indicate when the urine collecting assembly 502′ was disposed on the individual or to indicate other useful information. In an embodiment, the upper ring 578 may include a material that exhibits a color change at or around a certain time after being exposed to air which allows the upper ring 578 to indicate how long the urine collecting assembly 502′ has been in use.

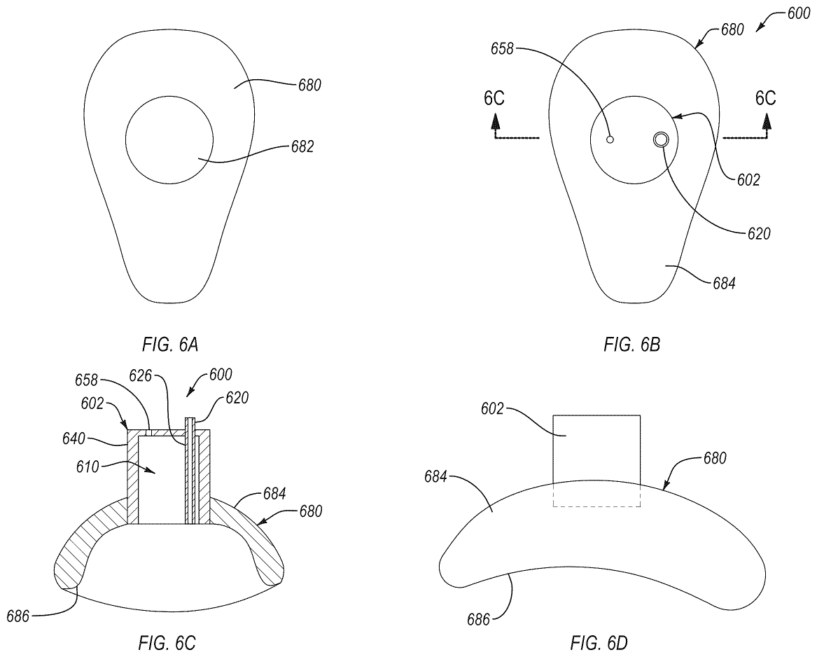

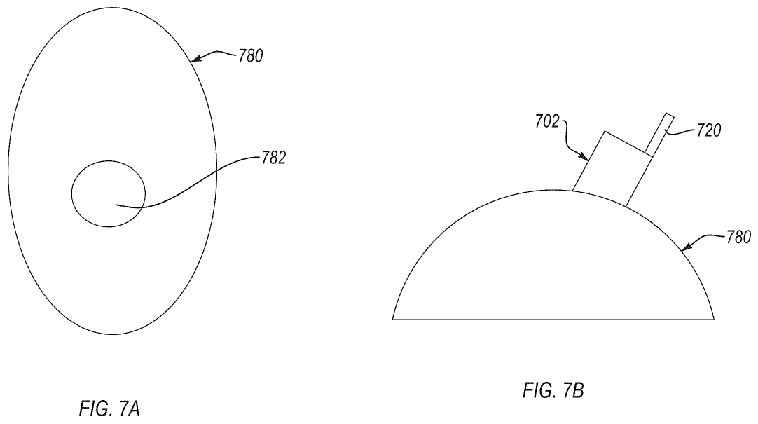

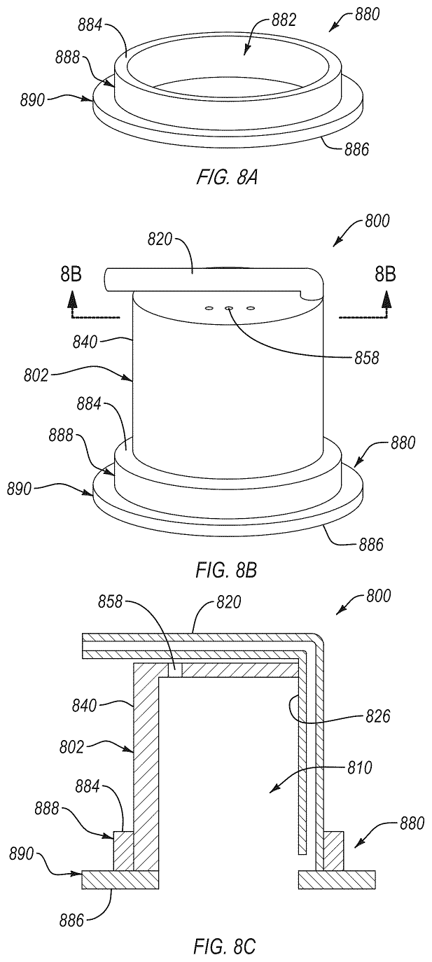

In an embodiment, a stabilizer or stabilization accessory can be used to maintain any of the urine collecting assemblies described herein in a particular position relative to a user's body. For example, FIG. 6A is a schematic top view of a stabilization accessory 680, according to an embodiment. The stabilization accessory 680 can be the same or similar in structure and/or function to the stabilization accessory 180 described above with reference to FIG. 1. As shown in FIG. 6A, the stabilization accessory 680 defines an opening 682 configured to receive a urine collecting assembly 602 (shown in FIGS. 6B-6D), such as any of the urine collecting assemblies described herein. The size and shape of the opening 682 substantially corresponds to the size and shape of the urine collecting assembly 602, thereby preventing a fluid (e.g., urine) from flowing through the gap between the stabilization accessory 680 and the urine collecting assembly 602. The opening 682 can also extend completely through the stabilization accessory 680 thereby allowing a penis of a user of the stabilization accessory 680 to be fluidly coupled to a reservoir 610 (shown in FIG. 6C) of the urine collecting assembly 602. The stabilization accessory 680 can include a top surface 684 and an opposing bottom surface 686. The bottom surface 686 can be configured to contact a region about a user's penis.

The stabilization accessory 680 can be shaped and sized such that it can be disposed on a user's body (e.g., disposed about the user's penis). For example, the bottom surface 686 can exhibit a shape that substantially corresponds (e.g., substantially conforms) to a shape of region that is about the user's penis. In such an example, the bottom surface 686 can exhibit a concave curvature that substantially corresponds to the convex curvature of the region about the user's penis. In another example, at least a portion of the stabilization accessory 680 can be flexible such that the stabilization accessory 680 can be bent, flexed, or otherwise deformed to correspond to the shape of the region that is about the user's penis.

In an embodiment, the bottom surface 686 can include an adhesive (e.g., a hydrocolloid adhesive) that is configured to attach the stabilization accessory 680 to the user. The adhesive can also prevent the formation of gaps between the bottom surface 686 and the region about the user's penis when the user moves thereby preventing leaks between the region about the user's penis and the stabilization accessory 680.

The stabilization accessory 680 can maintain the urine collecting assembly 602 in a certain position and/or at a particular angle relative to a user's body (e.g., at an angle that is about 90°, less than about 90°, or greater than about 90° relative to an axis running along the length of a user lying supine). For example, in some situations of use, such as incontinence, disability that impairs mobility, restricted travel conditions (e.g., conditions experience by pilots, drivers, and/or workers in hazardous areas), monitoring, or for clinical testing, the stabilization accessory 680 can aid in maintaining the sealing engagement between the urine collecting assembly 602 and the user's penis. The stabilization accessory 680 can also enable the urine collecting assembly 602 to freely rotate within the opening 682, such as rotate in response to movement from the user. As previously discussed, allowing the urine collecting assembly 602 to rotate in the opening 682 can eliminate kinking, prevent links, and prevent pulling on the region about the user's penis while the user moves.

FIGS. 6B-6D show a top view, a front cross-sectional view, and a side view of a urine collecting system 600 that includes the stabilization accessory 680 engaged with the urine collecting assembly 602, according to an embodiment. The urine collecting assembly 602 can be the same or similar in structure and/or function to any of the urine collecting assemblies described herein. For example, the urine collecting assembly 602 can include a body 640 that defines a reservoir 610, an outlet 620 that can include an outlet tube 626, and one or more vacuum relief openings 658 formed in the body 640. In an embodiment, as shown in FIGS. 6B-6C, the urine collecting assembly 602 includes an outlet 620 extending from the top of the urine collecting assembly 602. In another embodiment (not shown), the urine collecting assembly 602 includes an outlet extending from the side of the urine collecting assembly 602. In such an embodiment, the stabilization accessory 680 can define a passageway through which a discharge line (not shown) can extend if the stabilization accessory 680 would otherwise at least partially obstruct the outlet.

Although shown in FIGS. 6A-6D as being a particular shape, the stabilization accessory can be any suitable shape and size. For example, FIG. 7A is a top view of a stabilization accessory 780 with an oblong shape, according to an embodiment. The stabilization accessory 780 can be the same or similar in structure and/or function to the stabilization accessory 180 or the stabilization accessory 580 described above. For example, the stabilization accessory 780 defines an opening 782 configured to receive a urine collecting assembly 702 (shown in FIG. 7B). The size and shape of the opening 782 substantially corresponds to the size and shape of the urine collecting assembly 702, thereby preventing a fluid (e.g., urine) from flowing through the gap between the stabilization accessory 780 and the urine collecting assembly 702. The opening 782 can also enable the urine collecting assembly 702 to rotate therein. In an embodiment, the stabilization accessory 780 can be secured to the user's body via, for example, adhesive tape or a hydrocolloid.

The stabilization accessory 780 can maintain the urine collecting assembly 702 in a certain position and/or at a particular angle relative to a user's body, such as an angle that is equal to, less than, or greater than about 90°. In an embodiment, maintaining the urine collecting assembly 702 in a certain position and/or a particular angle can aid in maintaining the sealing engagement between the urine collecting assembly 702 and the user's penis.

FIG. 7B is a side view of a urine collecting system 700 that includes the stabilization accessory 780 engaged with the urine collecting assembly 702, according to an embodiment. The urine collecting assembly 702 can be the same or similar in structure and/or function to any of the urine collecting assemblies described herein. The urine collecting assembly 702 includes an outlet 720 extending from the top of the urine collecting assembly 702.

FIG. 8A is an isometric view of a stabilization accessory 880, according to an embodiment. The stabilization accessory 880 can be the same or similar in structure and/or function to any of the stabilization accessories disclosed herein. The stabilization accessory 880 includes a raised portion 888 including a top surface 884 of the stabilization accessory 880 and a base portion 890 including a bottom surface 886 of the stabilization accessory 880. The raised portion 888 can extend upwardly from the base portion 890 and can exhibit an annular generally cylindrical shape. In an embodiment, the raised portion 888 is distinct from the base portion 890. In such an embodiment, the raised portion 888 can be attached to the base portion 890. In another embodiment, at least a portion of the raised portion 888 and at least a portion of the base portion 890 are integrally formed together.

The raised portion 888 and, optionally, the base portion 890 of the stabilization accessory 880 defines an opening 882. The opening 882 is configured to receive a urine collecting assembly 802 (shown in FIG. 8B). The size and shape of the opening 882 substantially corresponds to the size and shape of the urine collecting assembly 802 thereby preventing a fluid (e.g., urine) from flowing through a gap between the stabilization accessory 880 (e.g., the raised portion 888 and/or the base portion 890) and the urine collecting assembly 802. The opening 882 is also configured to enable the urine collecting assembly 802 to freely rotate therein.

The stabilization accessory 880 can be shaped and sized such that it can be disposed on a user's body (e.g., disposed about the user's penis). In an embodiment, the bottom surface 886 of the base portion 890 can exhibit a shape that corresponds to the region about the user's penis. In an embodiment, the base portion 890 can be flexible, thereby allowing the base portion 890 to be deformed such that the bottom surface 886 exhibits a shape that corresponds to the region about the user's penis. In an embodiment, the bottom surface 886 of the base portion 890 may exhibit a size (e.g., surface area) that allows the stabilization accessory 880 to be securely attached to the region about the user's penis. In an example, the bottom surface 886 may exhibit a surface area of at least about 5 cm² to about 10cm², about 7.5 cm² to about 15cm², about 10 cm² to about 20cm², about 15 cm² to about 25cm², about 20 cm² to about 30cm², about 25 cm² to about 35cm², about 30 cm² to about 40cm², about 35 cm² to about 45cm², about 40 cm² to about 50cm², about 45 cm² to about 60cm², about 50 cm² to about 70cm², about 60 cm² to about 80cm², about 70 cm² to about 90cm², about 80 cm² to about 100cm², about 90 cm² to about 120cm², about 110 cm² to about 140cm², about 130 cm² to about 160cm², about 150 cm² to about 200cm², or greater than about 200cm² . In an example, the bottom surface 886 may exhibit an dimension (e.g., diameter) of at most about 30 cm, at most about 25 cm, at most about 20 cm, at most about 15 cm, at most about 10 cm, or in ranges of about 10 cm to about 20 cm, about 15 cm to about 25 cm, or about 20 cm to about 30 cm. In an embodiment, the bottom surface 886 may exhibit a minimum width measured from an inner periphery of the bottom surface 886 to an outer periphery of the bottom surface 886 that is at most about 7.5 cm, at most about 5 cm, at most about 2.5 cm, at most about 1 cm, or in ranges of about 1 cm to about 3 cm, about 2 cm to about 4 cm, about 3 cm about 5 cm, about 4 cm to about 6 cm, or about 5 cm to about 7.5 cm. The size of the bottom surface 886 may depend on, for example, the strength of the attachment between the base portion 890 and the region about the users since increasing the size of the bottom surface 886 generally increases the attachment strength between the bottom surface 886 and the region about the user's penis.

The bottom surface 886 can include an adhesive that is configured to couple the stabilization accessory 880 to the region of the user about the user's penis. The adhesive can prevent the formation of gaps between the bottom surface 886 and the region about the user's penis even when the user moves. In other words, the base portion 890 can form an at least substantially fluid tight seal against the region about the user's penis. In an embodiment, the base portion 890 of the can include (e.g., consist of) a patch that includes the adhesive. For example, the base portion 890 can include a DuoDERM® patch or another suitable hydrocolloid patch.

FIGS. 8B and 8C is an isometric view and a schematic cross-sectional view, respectively, of a urine collecting system 800 that includes the stabilization accessory 880 engaged with an urine collecting assembly 802, according to an embodiment. The urine collecting assembly 802 can be the same or similar in structure and/or function to any of the urine collecting assemblies described herein. For example, the urine collecting assembly 802 can include a body 840 that defines a reservoir 810, and outlet 820 that includes an outlet tube 826, and one or more vacuum relief openings 858 formed in the body 840.

In an embodiment, as shown, the urine collecting assembly 802 does not include a sealing flange. Instead, the stabilization accessory 880 can form an at least substantially fluid tight seal against the region about the user's penis, thereby preventing urine from leaking from the system 800. The at least substantially fluid tight seal can enable urine that is discharged from the user's penis to pool at an intersection between the stabilization accessory 880 and the region about the user's penis substantially without leaking the urine from the urine collecting system 800 without contacting the penis. This allows the system 800 to be used with a penis that exhibit a diameter or a length that is insufficient to be used with the sealing flange. Further, the stabilization accessory 880 can also stabilize the urine collecting assembly 802 (e.g., maintain the correct position of the urine collecting assembly 802 relative to the penis) without contacting the penis. However, it is noted that the urine collecting assembly 802 can include a sealing flange thereby forming an additional mechanism to prevent urine from leaking from the system 800.

FIG. 8D is a schematic cross-sectional view of a urine collecting system 800′ that includes a stabilization accessory 880′ engaged with a urine collecting assembly 802′, according to an embodiment. Except as otherwise disclosed herein, the stabilization accessory 880′ can be the same as or similar to the stabilization accessory 880 of FIGS. 8A-8C and the urine collecting assembly 802′ is the same as or similar to the urine collecting accessory 880′ of FIGS. 8B-8C.

The stabilization accessory 880′ includes a first attachment mechanism 852A and the urine collecting assembly 802′ includes a second attachment mechanism 852B. The first attachment mechanism 852A and the second attachment mechanism 852B interact with each other in a manner that allows the urine collecting assembly 802′ to be securablely and reversibly coupled to the stabilization accessory 880′. In an embodiment, as illustrated, the first attachment mechanism 852A includes at least one protrusion extending from an internal surface 889 of the stabilization accessory 880′. Similarly, the second attachment mechanism 852B includes a protrusion extending from an external surface 854 of the body 840′. The first and second attachment mechanisms 852A, 852B reversibly couple the urine collecting assembly 802′ to the stabilization accessory 880′ because the first attachment mechanism 852A defines an internal diameter of the stabilization accessory 880′ that is smaller than an external diameter of the urine collecting assembly 802′ defined by the second attachment mechanism 852B. As such, coupling and decoupling the urine collecting assembly 802′ from the stabilization accessory 880′ can require an external force applied thereto before the second attachment mechanism 852B slides over the first attachment mechanism 852A.

It is noted that at least one of the first or second attachment mechanism 852A, 852B can be include any of the attachment mechanisms disclosed herein. For example, one of the first or second attachment mechanism 852A, 852B can include at least one protrusion and the other of the first or second attachment mechanism 852A, 852B can include at least one recess that is configured to receive the protrusion. In another example, the first and second attachment mechanisms 852A, 852B can include threads. In an embodiment, at least one of the first or second attachment mechanism 852A, 852B may be omitted from the stabilization accessory 880′, as shown in FIG. 8C. In such an embodiment, the urine colleting assembly 802′ may be maintained in the stabilization accessory 880′ via friction.

The stabilization accessories disclosed herein, up to this point, exhibit a single piece construction or are formed of two or more pieces that are permanently coupled together. However, in some embodiments, any of the stabilization accessories disclosed herein can be formed from two or more pieces, such as a first piece and a second piece, that are reversibly coupled together. Forming the stabilization accessories from two or more pieces has several benefits. For example, one of the two or more pieces (e.g., the first piece) can be semi-permanently coupled to a user and another of the two or more pieces (e.g., the second piece) can be temporarily coupled to the user via the first piece. This allows portions of the stabilization accessory to be decoupled from the user to examine underlying anatomy, be disposed of, replaced the when damaged, etc. In another example, forming the stabilization accessory from two or more pieces can allow portions of the stabilization accessory to be removed when a fluid is not actively being removed from the individual thereby decreasing the profile of the stabilization accessory and allowing a user to wear portions of the stabilization accessory more discretely.

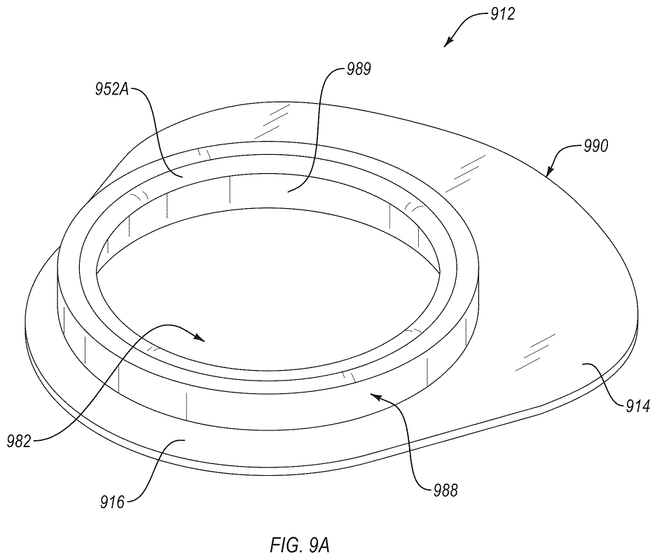

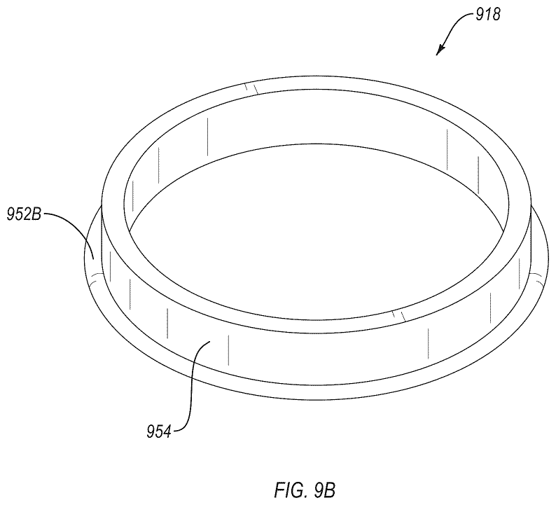

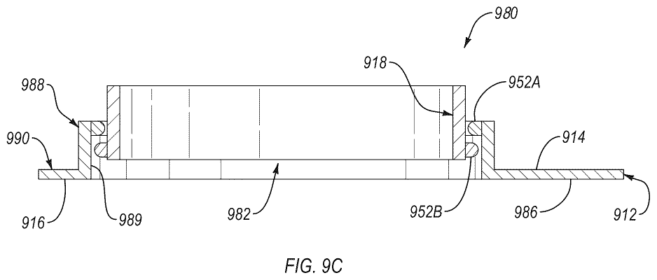

FIG. 9A is an isometric view of a first piece 912 of a stabilization accessory 980 (shown assembled in FIG. 9C), according to an embodiment. The first piece 912 includes a base portion 990 defining an opening 982 and a raised portion 988 extending from a portion of the base portion 990 defining the opening 982. Except as otherwise disclosed herein, the first piece 912 can be formed of any of the same or similar materials as, exhibit any of the same or similar properties of, or exhibit any of the elements of any of the stabilization accessories disclosed herein. For example, the first portion 912 can be formed of a flexible material, can include an adhesive on a bottom surface 986 (shown in FIG. 9C), etc.

The base portion 990 can exhibit any suitable shape. In an embodiment, the base portion 990 exhibits a rounded generally triangular shape, as shown in FIG. 9A. The opening 982 can be located on one side of the base portion 990 near one of the vertices of the base portion 990. This causes the base portion 990 to exhibit a primary attachment portion 914 that is on the side of the base portion 990 that is opposite the opening 982 and a secondary attachment portion 916 that surrounds the opening 982. The primary attachment portion 914 exhibits a surface area that is significantly larger than the secondary attachment portion 916. As such, the primary attachment portion 914 can be configured to couple the base portion to less sensitive regions of the user (e.g., a public mound) while the secondary attachment portion 914 can be configured to be coupled to more sensitive regions of the user (e.g., around the penis, the scrotum, or the perineal region). It is noted that any of the stabilization accessories disclosed herein can exhibit the rounded generally triangular shape of the base portion 990 and/or the openings thereof can be off centered thereby forming a primary and second attachment portions.

The raised portion 988 can be configured to be reversibly coupled to a second piece 918 (shown in FIGS. 9B and 9C). For example, the raised portion 988 can include at least one first attachment mechanism 952A on an internal surface 989 thereof. The first attachment mechanism 952A can include any of the attachment mechanisms 952A disclosed herein, such as at least one protrusion.

FIG. 9B is an isometric view of the second piece 918 of the stabilization accessory 980 (shown assembled in FIG. 9C), according to an embodiment. Except as otherwise disclosed herein, the second piece 918 can be formed of any of the same or similar materials as, exhibit any of the same or similar properties of, or exhibit any of the elements of any of the stabilization accessories disclosed herein.

The second piece 918 exhibits a shape that substantially corresponds to the shape of the raised portion 988 of the first piece 912. The second piece 918 also exhibits a cross-sectional dimension (e.g., diameter) that is slightly smaller than a cross-sectional dimension of the opening 982 of the second piece 912. As such, the shape and the cross-sectional dimension of the second piece 918 allows the second piece 918 to fit within the first piece 912. However, the second piece 918 can exhibit a height measured perpendicularly to the cross-sectional dimension that is greater than the raised portion 988 of the first piece 912. The larger height of the second piece 918 can allow the second piece 918 to extend above the raised portion 988 of the first piece 912.

The second piece 918 also include a second attachment mechanism 952B on an external surface 954 thereof. The second attachment mechanism 952B is configured to interact with the first attachment mechanism 952B of the first piece 918, thereby allowing the second piece 918 to be reversibly coupled to the first piece 912. The second attachment mechanism 952B can include any of the attachment mechanisms disclosed herein. For example, as illustrated, the second attachment mechanism 952B can include at least one protrusion. In an embodiment, the first and second attachment mechanisms 952A, 952B are configured to allow the second piece 918 to rotate relative to the first piece 918.

FIG. 9C is a schematic cross-sectional view of the assembled stabilization accessory 980, according to an embodiment. As illustrated, the second piece 918 can be disposed within the raised portion 988 of the second piece 912. However, the second piece 918 exhibits a height that is greater than the raised portion 988 of the first piece 912 thereby allowing the second piece 918 to extend above the raised portion 988. The first and second attachment mechanisms 952A, 952B also reversibly couple the first and second pieces 912, 918 together. However, the first and second attachment mechanisms 952A, 952B can allow the first and second pieces 912, 918 to be decoupled from each other when a force is applied thereto that is sufficient to elastically deform the first and second pieces 912, 918 to an extent that allows the first and second pieces 912, 918 to be decoupled.

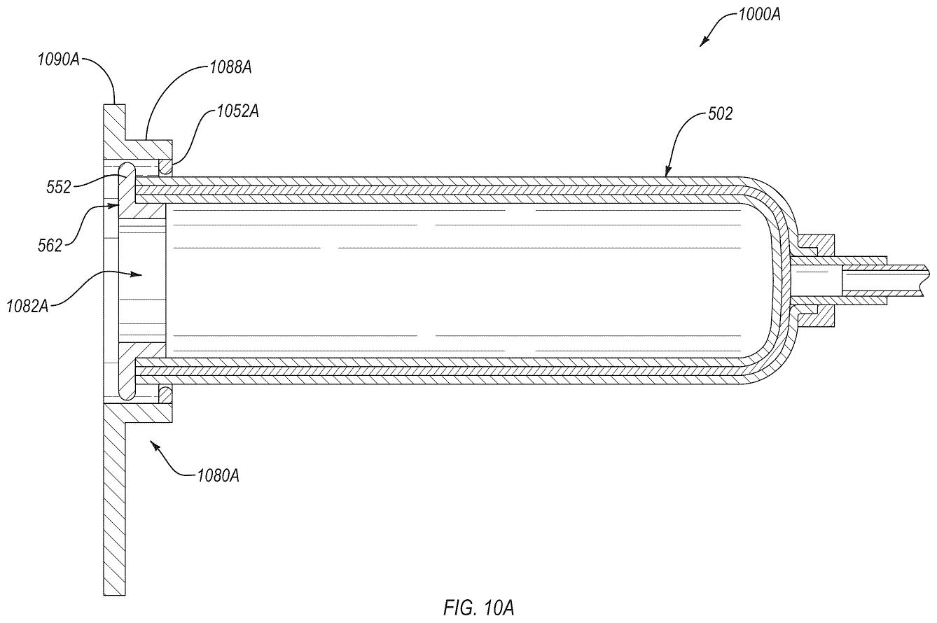

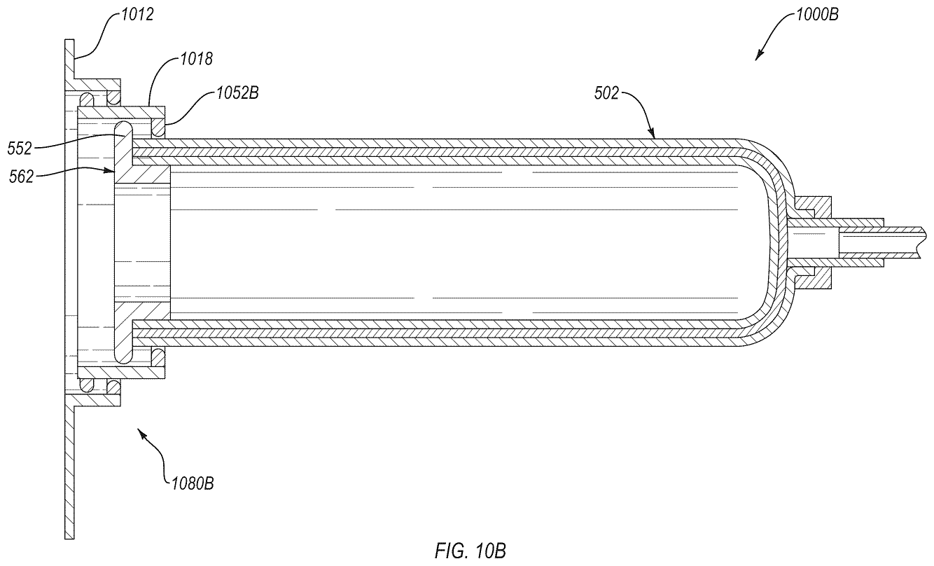

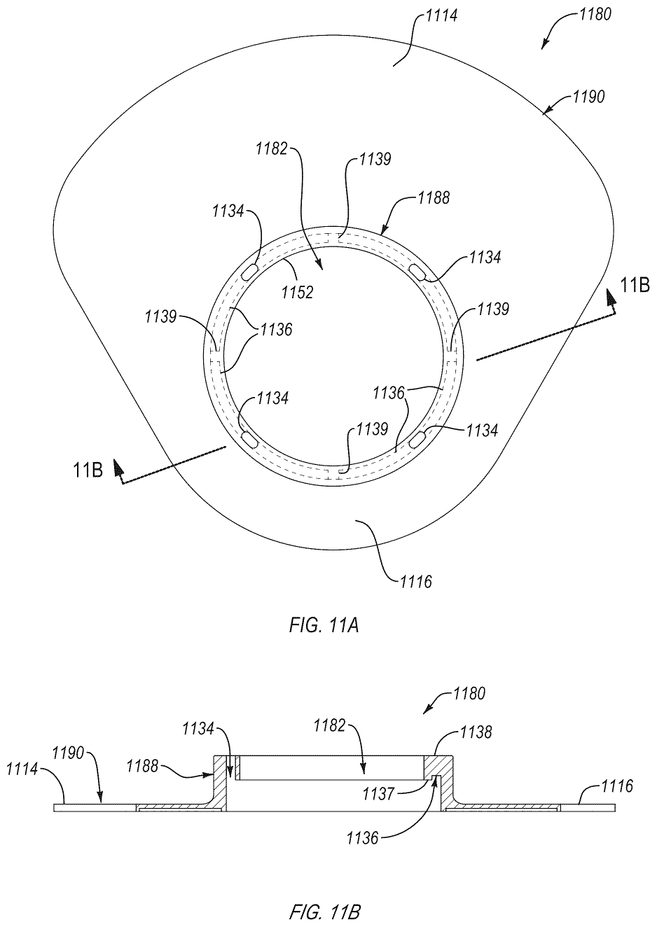

It is noted that the second piece 918 can be configured to have any of the urine collecting assemblies disclosed herein disposed therein. For example, the second piece 918 can include an additional attachment mechanism that is configured to reversibly couple one of the urine collecting assemblies therein (as shown in FIG. 10B).

The stabilization accessories disclosed herein can also be configured to have the urine collecting assembly of FIGS. 5A-5B disposed therein. For example, FIG. 10A is a schematic cross-sectional view of a system 1000A that includes the urine collecting assembly 502 of FIGS. 5A-5B disposed in a stabilization accessory 1080A, according to an embodiment. The stabilization accessory 1080A can include any of the stabilization accessories disclosed herein. For example, the stabilization accessory 1080A can include a base portion 1090A defining an opening 1082A and a raised portion 1088A. The opening 1082A can be off centered similar to the opening 982 of FIG. 9A. In an embodiment, the stabilization accessory 1080A can also include an attachment mechanism 1052A that is configured to interact with the ring 562 of the urine collecting assembly 502 (e.g., a protrusion of the ring 562), thereby reversibly coupling the urine collecting assembly 502 with the stabilization accessory 1080A. However, at least one of the attachment mechanism 1052A can be omitted from the stabilization accessory 1080A or the protrusion can be omitted from the ring 562.

FIG. 10B is a schematic cross-sectional view of a system 1000B that include the urine collecting assembly 502 of FIGS. 5A-5B disposed in a stabilization accessory 1080B, according to an embodiment. Except as otherwise disclosed herein, the stabilization accessory 1080B can be the same as or similar to the stabilization accessory 980 of FIG. 9C. For example, the stabilization accessory 1080B includes a first piece 1012 and a second piece 1018 reversibly coupled together. However, the second piece 1018 includes an attachment mechanism 1052B that is configured to interact with the ring 562 of the urine collecting assembly 502 thereby reversibly coupling the urine collecting assembly 502 with the stabilization accessory 1080B. However, at least one of the attachment mechanism 1052B can be omitted from the second piece 1018 or the protrusion can be omitted from the ring 562.

It is noted that, in an embodiment, the system 1000B can include any of the urine collecting assemblies disclosed herein instead of the urine collecting assembly 502.

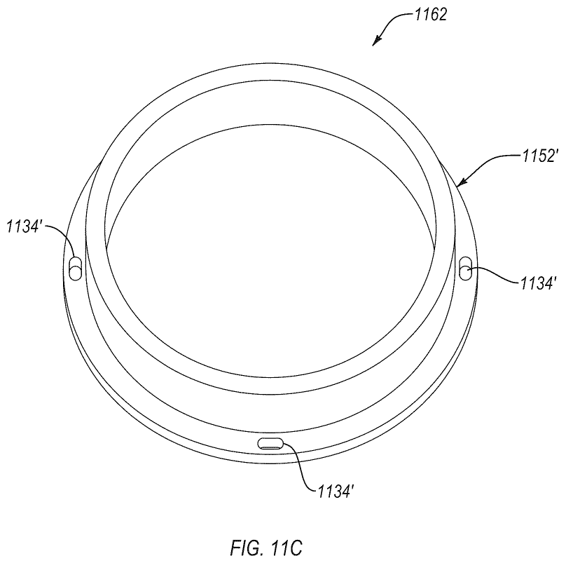

FIGS. 11A and 11B are a top plan view and a cross-sectional view, respectively, of a stabilization accessory 1180, according to an embodiment. Except as otherwise disclosed herein, the stabilization accessory 1180 may be the same as or substantially similar to any of the stabilization accessories disclosed herein. For example, the stabilization accessory 1180 may include a base portion 1190 and a raised portion 1188. The base portion 1190 may include a primary attachment portion 1114 and a secondary attachment portion 1116. The raised portion 1118 may include a first attachment mechanism 1152 (e.g., protrusion) extending circumferentially inwardly relative to the rest of the raised portion 1118. The first attachment mechanism 1152 may be integrally formed with the rest of the raised portion 1118 (as shown) or may be separate and distinct from the rest of the raised portion 1118 (as shown in FIG. 8D).

The stabilization accessory 1180 defines one or more vents 1134 extending therethrough. The vents 1134 may allow air to flow through the stabilization accessory 1180 which may reduce discomfort and/or bruises (e.g., hickies) caused by a vacuum source. For example, during operation, the vents 1134 may allow the air to flow from an exterior of a urine collecting system that includes the stabilization accessory 1180 and into the reservoir of the urine collecting system.

In an embodiment, as illustrated, the vents 1134 may be formed in and extend through the first attachment mechanism 1152. Forming the vents 1134 in the first attachment mechanism 1152 may limit bodily fluid flow through the vents 1134 since any bodily fluid reaching the vents 1134 would have to flow in a tortuous path (e.g., flow between an interior surface 1189 of the stabilization accessory and an exterior surface of the urine collecting assembly) and against the air flow flowing through the tortuous path.

When the vents 1134 are formed in the first attachment mechanism 1152 of the stabilization accessory 1180, the vents 1134 may be at least partially covered by a corresponding second attachment mechanism 1152′ of the urine collecting assembly (shown in FIG. 11C). To prevent the corresponding second attachment mechanism 1152′ of the urine collecting assembly from covering and obstructing the flow of air through the vents 1134, the raised portion 1188 may define at least one recess 1136 therein. The recess 1136 is shown are shown in FIG. 11A using dashed lines and in FIG. 11B.

Referring to FIG. 11B, which shows a vent 1134 on one side of the opening 1182 of the stabilization accessory 1180 and a recess 1136 on the other side of the opening 1182, the recess 1136 may extend inwardly from a bottom surface 1137 of the first attachment mechanism 1152 towards a top surface 1137 of the first attachment mechanism 1152 for a distance that is less than the thickness of the first attachment mechanism 1152 (e.g., measured from the bottom surface 1137 to an opposing top surface 1138 of the first attachment mechanism 1152). For example, the recess 1136 may extend for a distance that less than half the thickness of the first attachment mechanism 1152 since limiting the distance that the recess 1136 extends into the first attachment mechanism 1152 may limit the amount of bodily fluid that may flow therethrough

Referring to FIG. 11A, in an embodiment, the recess 1134 may include a plurality of recesses 1134. In such an embodiment, the recess 1134 may include one or more transverse supports 1139 (shown with dashed lines) that at least partially separate each of the plurality of recesses 1134 from each other. The transverse supports 1139 are portions of the first attachment mechanism 1152 circumferentially between the recesses 1136 that exhibit a thickness that is greater than a thickness of portions of the first attachment mechanism 1152 measured between the recesses 1134 and the top surface 1138. The transverse supports 1139 may at least one of improve the strength of the first attachment mechanism 1152 (e.g., prevent folding of the attachment mechanism along the plurality of recesses 1134) or prevent bodily fluid that reach one of the recesses 1134 from leaking into another one of the recesses 1134, thereby limiting bodily fluid leaks through multiple vents 1134.

FIG. 11C is an isometric view of a second attachment mechanism 1152′ of a urine collecting assembly (not shown for clarity) that is configured to be used in conjunction with the stabilization accessory 1180 of FIGS. 11A and 11B, according to an embodiment. Except as otherwise disclosed herein, the second attachment mechanism 1152′ may be the same or substantially similar to any of the attachment mechanisms of the urine collecting assemblies disclosed herein. For example, the second attachment mechanism 1152′ may form part of a ring 1162 (as illustrated), a second piece (e.g., second piece 912), or a body (e.g., body 840′).

The second attachment mechanism 1152′ defines one or more vents 1134′ extending therethrough. The vents 1134′ may allow the air to flow through the second attachment mechanism 1152′ and reach the vents 1134 of the first attachment mechanism 1152 when, for example, the gap between the second attachment mechanism 1152′ and the raised portion 1188 of the stabilization accessory 1180 is sufficiently small to inhibit sufficient air flow therethrough.

In an embodiment, the second attachment mechanism 1152′ may include the same number of vents 1134′ that are positioned to correspond to the location of the vents 1134 of the first attachment mechanism 1152. However, in another embodiment, the second attachment mechanism 1152′ may include a different number of vents 1134′ than the first attachment mechanism 1152 and/or the vents 1134′ may not be positioned to correspond to the vents 1134 of the first attachment mechanism 1152.

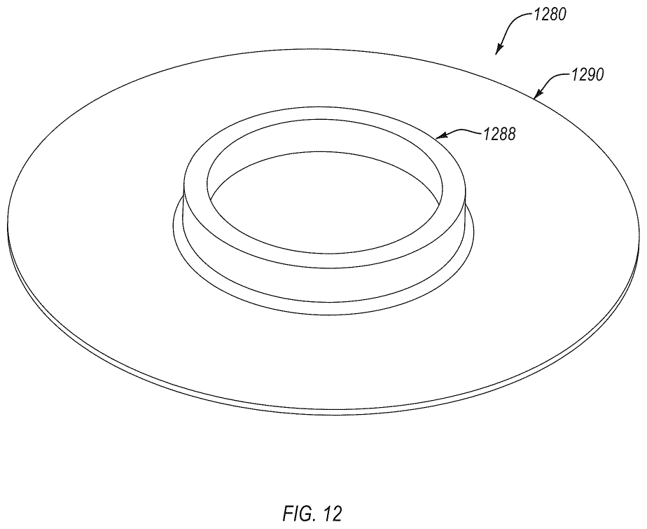

FIG. 12 is a top plan view of a stabilization accessory 1280, according to an embodiment. Except as otherwise disclosed herein, the stabilization accessory 1280 may be the same or substantially similar to any of the stabilization accessories disclosed herein. For example, the stabilization accessory 1280 may include a raised portion 1288 and a base portion 1290.

Unlike some of the stabilization accessories disclosed herein, the raised portion 1288 is centrally located on the base portion 1290. Further, the base portion 1290 may extend radially equidistantly from the raised portion 1288. As such, the stabilization accessory 1280 may be disposed on a user without regard to properly rotating the stabilization accessory 1280 relative to the user. For example, the stabilization accessory 1180 illustrated in FIG. 11A may need to be properly rotated relative to the user such that the primary attachment portion 1114 contacts the pubic region (e.g., the lower part of the abdomen just above the penis) of the user to effectively attach the stabilization accessory 1180 to the user. Meanwhile, referring back to FIG. 12, the stabilization accessory 1280 has no such requirement. However, the size (e.g., surface area) of the base portion 1290 may be limited by the distance between the penis and the hip of the user and, as such, the base portion 1290 may exhibit a size that is less than the size of the base portion 1190 of the stabilization accessory 1190 illustrated in FIG. 11A.

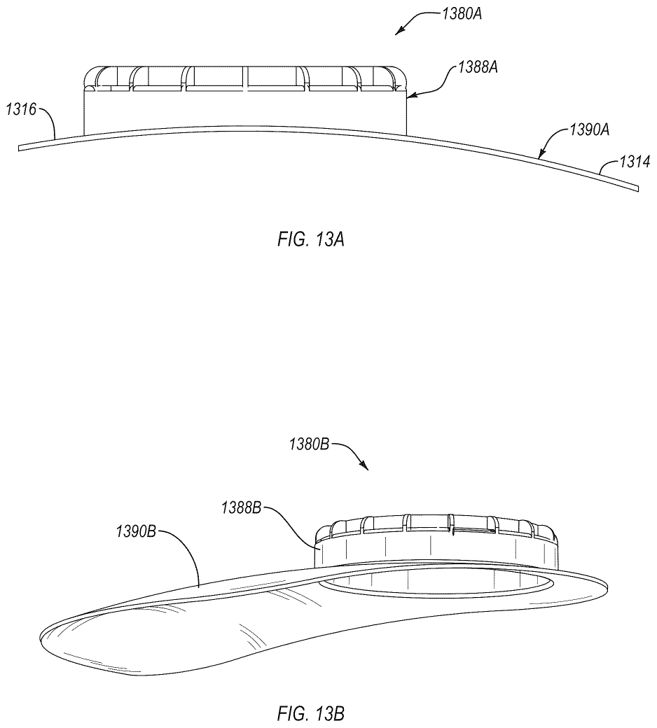

FIG. 13A is a side plan view of a stabilization accessory 1380A, according to an embodiment. Except as otherwise disclosed herein, the stabilization accessory 1380A may be the same as or substantially similar to any of the stabilization accessories disclosed herein. For example, the stabilization accessory 1380A may include a raised portion 1388A and a base portion 1390A.

The base portion 1390A may exhibit a contour (e.g., curve) along a single axis. The single axis may extend, for example, longitudinally from the primary attachment portion 1314 to a secondary attachment portion 1316, wherein the single axis may extend radially across an opening (not shown, obscured) of the stabilization accessory 1380A. Contouring the base portion 1390A may facilitate attachment of the base portion 1390A to a user since the area around the penis of the user exhibits some curvature. Further, contouring the base portion 1390A may improve the comfort of wearing the stabilization accessory 1380A since the stabilization accessory 1380A may pull less on the regions about the penis of the user than a substantially similar stabilization accessory 1380A that includes a planar base portion.

FIG. 13B is a side plan view of a stabilization accessory 1380B, according to an embodiment. Except as otherwise disclosed herein, the stabilization accessory 1380B may be the same as or substantially similar to any of the stabilization accessories disclosed herein. For example, the stabilization accessory 1380B may include a raised portion 1388B and a base portion 1390B.

The base portion 1390B may exhibit a first contour (e.g., curve) along a first axis similar to the base portion 1390A of FIG. 13A exhibiting a contour along the single axis. However, the base portion 1390B may also exhibit at least one addition contour (e.g., a second contour) along at least one addition axis (e.g., a second axis) that is transverse (e.g., perpendicular or obliquely angled) relative to the first axis. Contouring the base portion 1390B along the first axis and the at least one additional axis may allow the base portion 1390B to more closely conform to the shape of the regions about the penis of the user than a substantially similar stabilization accessory that includes a planar base portion or a base portion that is contoured along a single access. The more closely the base portion 1390B conforms to the region about the penis of the user may better facilitate attachment and/or improve comfort of wearing the stabilization accessory 1380B.

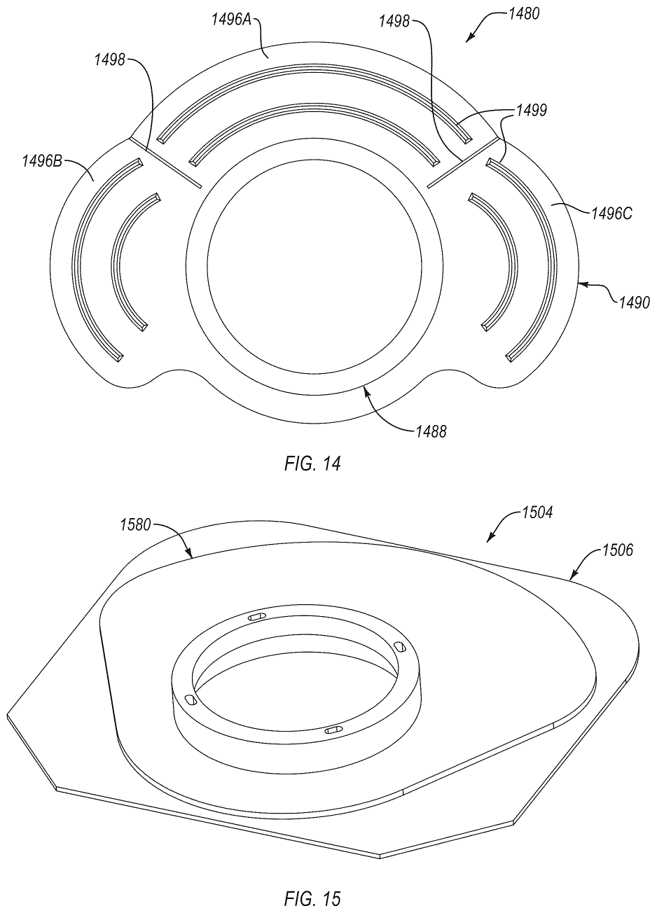

FIG. 14 is a top plan view of a stabilization accessory 1480, according to an embodiment. Except as otherwise disclosed herein, the stabilization accessory 1480 may be the same or substantially similar to any of the stabilization accessories disclosed herein. For example, the stabilization accessory 1480 may include a raised portion 1488 and a base portion 1490.

The base portion 1488 includes a plurality of sections that are configured to attach to different regions of the user. Since each of the plurality of sections are configured to be attached to different regions of the user, at least one of the plurality of sections may exhibit different shapes than another one of the plurality of regions. The different shapes of the plurality of sections may cause the base portion 1488 to exhibit an irregular shape. For example, as illustrated, the base portion 1488 may include a first portion 1496A, a second portion 1496B, and a third portion 1496C. The first portion 1496A may be configured to be attached to a pubic region of a user while the second and third portions 1496B and 1496C may be configured to be attached to the hips and/or a region of the user extending from the penis to the hips of the user. As such, the first portion 1496A may exhibit a first shape and the second and third portions 1496B and 1496C may exhibit a second shape that is different than the first shape. For example, each of the first, second, and third portions 1496A, 1496B, and 1496C may exhibit generally arcuate shapes. However, the arcuate shape of the first portion 1496A may exhibit a larger radius than the arcuate shape of the second and third portions 1496B and 1496C.

In an embodiment, as illustrated, the first, second, and third portions 1496A, 1496B, and 1496C may be separated from each other by a slit 1498 which may allow the first, second, and third portions 1496A, 1496B, and 1496C to bend independently from each other. However, it is noted that the base portion 1490 may include a flexible and/or elastic material between the first, second, and third portions 1496A, 1496B, and 1496C which allows the first, second, and third portions 1496A, 1496B, and 1496C to bend independently from each other or the first, second, and third portions 1496A, 1496B, and 1496C may be integrally formed with each other.

In an embodiment, at least one of the first, second, or third portions 1496A, 1496B, or 1496C may include one or more ridges 1499 formed thereon. The ridges 1499 may facilitate gripping of the first, second, or third portions 1496A, 1496B, or 1496C which may facilitate bending of the first, second, or third portions 1496A, 1496B, or 1496C.

The stabilization accessories disclosed herein may be attached to an individual using any suitable method. In an embodiment, the stabilization accessories disclosed herein may include an adhesive (e.g., hydrogel) on a bottom surface thereof that is configured to attach the stabilization accessory to the user. FIG. 15 is an isometric view of packaging 1504 that includes a stabilization accessory 1580 that is configured to be adhesively attached to an individual and a pad 1506, according to an embodiment. The stabilization accessory 1580 may include any of the stabilization accessories disclosed herein. The stabilization accessory 1580 may be attached to the pad 1506 using an adhesive (not shown). The stabilization accessory 1580 and the pad 1506 may be configured such that the stabilization accessory 1580 may be easily remove from the pad 1506 when the stabilization accessory 1580 is attached to the user.

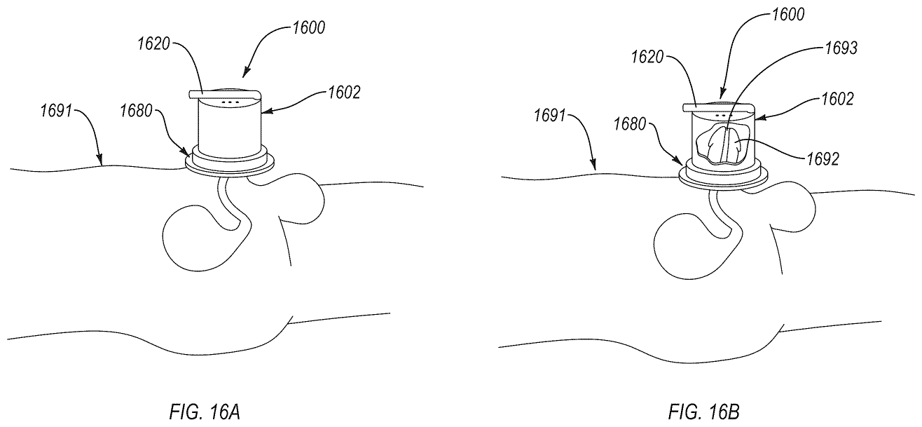

FIGS. 16A and 16B are a schematic view and a schematic cut-away view, respectively of a urine collecting system 1600 disposed on a user 1691 in a position for use, according to an embodiment. The urine collecting system 1600 can be the same or similar in structure and/or function to any of the urine collecting systems disclosed herein. For example, the urine collecting system 1600 can include a urine collecting assembly 1602 and a stabilization accessory 1680 that is the same or similar in structure and/or function to any of the urine collecting assemblies and stabilization accessories, respectively, disclosed herein. For example, the urine collecting assembly 1602 includes an outlet 1620.

In use, as shown in FIGS. 16A and 16B, the urine collecting system 1600 can be positioned such that the urine collecting assembly 1602 encloses a portion of the penis 1692 of the user 1691. For example, the urine collecting assembly 1602 can be positioned such that the urethral opening 1693 of the user 1691 is within a body of the urine collecting assembly 1602 and, optionally, a sealing flange (not shown) of the urine collecting assembly 1602 sealingly engages a shaft of the user's penis 1692. In such a position, urine can exit the urethral opening 1693 of the penis 1692 and collect in a reservoir (due to gravity) defined by at least a portion of the user 1691 (e.g., the outer surface of the shaft of the penis 1692) and a side wall of the urine collecting assembly 1602. A vacuum source (e.g., vacuum source 170) can be used to draw the collected urine from the outlet 1620. In urine collecting assemblies having an outlet positioned near the bottom of the urine collecting assembly, gravity and/or a vacuum source can cause or assist the travel of urine from the urine collecting assembly 1602 via the outlet. The vacuum source can be fluidly coupled to an external receptacle via a vacuum line such that gaseous fluid is drawn from the external receptacle via the vacuum line. As a result of the decrease in pressure within the external receptacle caused by the drawing of gaseous fluid out of the external receptacle, liquid and/or gaseous fluid can be drawn from the reservoir, through the outlet 1620, through a discharge line, and into the external receptacle.

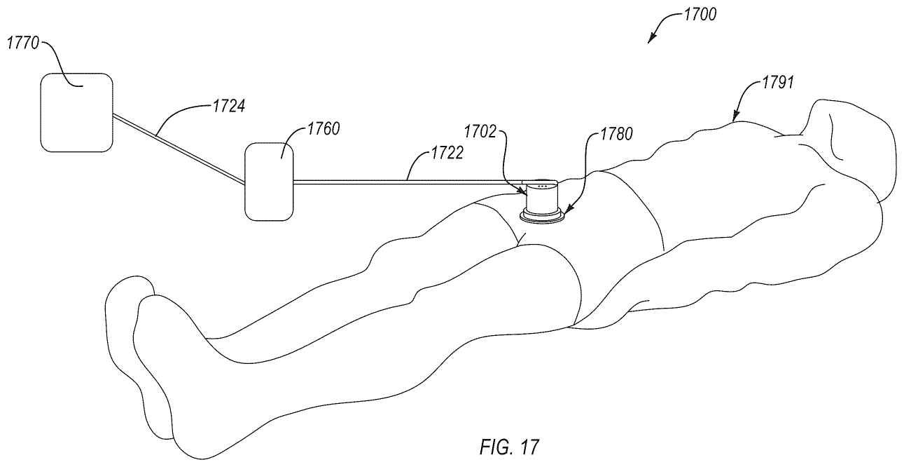

FIG. 17 is a schematic illustration of a urine collecting system 1700 disposed on the body of a user 1791, according to an embodiment. The urine collecting system 1700 can be the same or similar in structure and/or function to any of the urine collecting systems disclosed herein. The urine collecting system 1700 includes a urine collecting assembly 1702 and a stabilization accessory 1780 that can be the same or similar in structure and/or function to any of the urine collecting assemblies and stabilization accessories, respectively, described herein. The urine collecting system 1700 can include an external receptacle 1760 and a vacuum source 1770. The external receptacle 1760 can be the same or similar in structure and/or function as the external receptacle 160 described above. The vacuum source 1770 can be the same or similar in structure and/or function as the vacuum source 170 described above. The urine collecting assembly 1702 can be fluidly coupled to the external receptacle 1760 via a discharge line 1722. The external receptacle 1760 can be coupled to the vacuum source 1770 via a vacuum line 1724.

In use, as shown in FIG. 17, the urine collecting system 1700 can be positioned such that the urine collecting assembly 1702 encloses a portion of the penis of the user 1781. For example, the urine collecting assembly 1702 can be positioned such that the urethral opening of the user 1791 is within a body of the urine collecting assembly 1702 and, optionally, a sealing flange of the urine collecting assembly 1702 sealingly engages a shaft of the user's penis. In such a position, urine can exit the urethral opening of the penis and collect in a reservoir (due to gravity) defined by user 1791 and a side wall of the urine collecting assembly 1702.

Similarly as described above with reference to urine collecting system 100, the vacuum source 1770 can assist and/or provide the pressure differential needed to draw fluid (e.g., urine) voided from the urethral opening into the reservoir of the urine collecting assembly 1702 from the reservoir, through the discharge line 1722, and into the external receptacle 1760. More specifically, the vacuum source 1770 can be fluidly coupled to the external receptacle 1760 via the vacuum line 1724 such that gaseous fluid can be drawn from the external receptacle 1760 via the vacuum line 1724. As a result of the decrease in pressure within the external receptacle 1760 caused by the drawing of gaseous fluid out of the external receptacle 1760, liquid and/or gaseous fluid can be drawn from the reservoir of the urine collecting assembly 1702, through the outlet 1720, through the discharge line 1722, and into the external receptacle 1760.

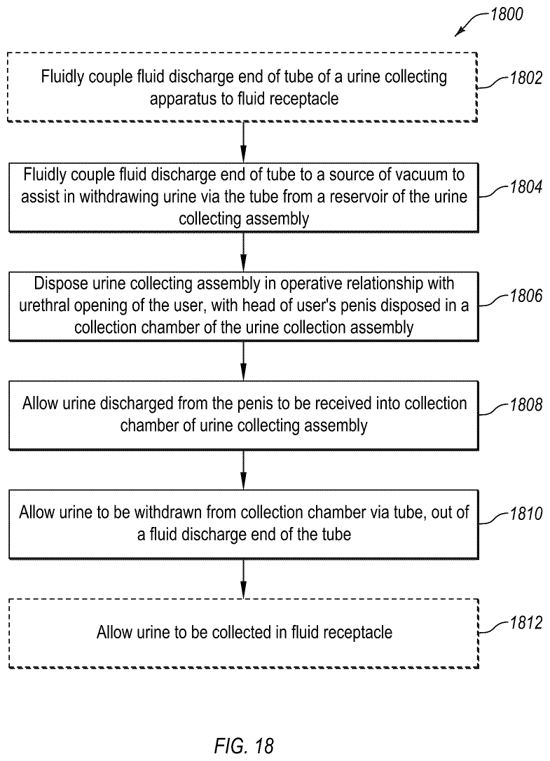

FIG. 18 is a flowchart illustrating a method 1800 of using a urine collecting assembly to collect urine from a user, according to an embodiment. The method 1800 can include at least some of acts 1802, 1804, 1806, 1808, 1810, or 1812. The method 1800 is for illustrative purposes and, as such, at least one of the acts 1802, 1804, 1806, 1808, 1810, or 1812 can be performed in a different order, split into multiple acts, modified, supplemented, combined, or omitted.

The method 1800 optionally includes, at act 1802, fluidly coupling a discharge end of a tube of a urine collecting assembly to a fluid receptacle. Method 1800 optionally further includes, at act 1804, fluidly coupling the discharge end of the tube of the urine collecting urine collecting assembly to a source of vacuum.

Method 1800 further includes, at act 1806, disposing the urine collecting urine collecting assembly in operative relationship with the urethral opening of the user, with a head of a penis of a male user (e.g. human or animal) disposed in a reservoir of the urine collecting assembly (e.g., through a sealing flange). The urine collecting assembly can be the same or similar in structure and/or function to any of the urine collecting assemblies described herein. For example, the urine collecting assembly can include at least one of a body, a sealing flange, or a reservoir within the body and partially defined by the sealing flange. The sealing flange can define an opening such that the interior of the body is accessible via the opening. A peripheral edge of the opening can be configured to seal around a shaft of a penis of a user disposed through the opening. The urine collecting assembly can also include an outlet in fluid communication with the reservoir. The urine collecting assembly can be arranged such that a fluid can flow into the body from the urethral opening of the user's penis, collect in the reservoir, and flow out of the outlet.

In an embodiment, the urine collecting assembly forms part of a urine collecting urine collecting system that includes a stabilization accessory. The stabilization accessory can be the same or similar in structure and/or function to any of the stabilization accessories disclosed herein. For example, the stabilization accessory can define an opening that is configured to receive the urine collecting assembly and allow the urine collecting assembly to rotate therein. In such an embodiment, act 1806 can include disposing the stabilization accessory on a region about the user's penis and disposing the urine collecting assembly in the opening of the stabilization accessory. In an embodiment, disposing the stabilization accessory on a region about the user's penis includes attaching (e.g., using an adhesive) a bottom surface of the stabilization accessory to the region about the user's penis.

The method 1800 also includes, at 1808, allowing urine discharged from the penis to be received into the reservoir of the urine collecting assembly.

The method 1800 also includes, at act 1810, allowing the received urine to be withdrawn from the reservoir via the tube and out of the fluid discharge end of the tube.

The method 1800 optionally includes, at act 1812, removing the urine collecting assembly from the penis of the user.

The method 1800 optionally includes disposing a second urine collecting assembly in operative relationship with the urethral opening of the user, with the head of the penis of the user disposed through the sealing flange and into the reservoir of the urine collecting assembly.

In an embodiment, the user can move while the urine collecting assembly and the stabilization accessory is disposed around the user's penis. In such an embodiment, the method 1800 can include rotating the urine collecting assembly in the opening of the stabilization accessory responsive to the movement of the user.

While various embodiments of the urine collecting system, methods and devices have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, although various embodiments have been described as having particular features and/or combinations of components, other embodiments are possible having any combination or sub-combination of any features and/or components from any of the embodiments described herein. In addition, the specific configurations of the various components can also be varied. For example, the size and specific shape of the various components can be different than the embodiments shown, while still providing the functions as described herein.

1. (canceled)

2. A urine collecting system, comprising:
a urine collecting assembly including a body, the body including a proximal end and a distal end, the body defining a first opening at or near the proximal end, the body including:
a sheath extending from or near the proximal end to or near the distal end, the sheath including at least one fluid impermeable layer, the sheath at least partially defining a reservoir; and
an outlet at or near the distal end of the body, the outlet is distinct and separate from the at least one fluid impermeable layer, the outlet attached to the at least one fluid impermeable layer; and
at least one porous material extending from or near the outlet to the proximal end, the at least one porous material adjacent to the fluid impermeable layer;
a stabilization accessory defining a second opening therethrough, the stabilization accessory comprising:
a base portion including a primary attachment portion and a secondary attachment portion, the base portion exhibits a maximum width measured perpendicular to a longitudinal axis of the body, and wherein the primary attachment portion includes at least a portion of the maximum width, wherein the secondary attachment portion includes an apex forming the minimum width of the stabilization accessory and the minimum width is spaced from and on an opposite side of the second opening from at least a portion of the primary portion including the maximum width; and
a first attachment mechanism configured to attach the stabilization accessory to the body; and
wherein the reservoir is defined by at least the outlet, the at least one porous material, and the fluid impermeable layer, such that the porous material forms a fluid path between the first opening and the reservoir.

3. The urine collecting system of claim 2, wherein the distal end of the body is an at least partially closed distal end.

4. The urine collecting system of claim 2, wherein a portion of the sheath at or near the distal end defines a hole; and wherein the urine collecting assembly further comprises a top porous layer that at least partially covers the hole.

5. The urine collecting system of claim 2, wherein the body includes a sump, the sump including the outlet.

6. The urine collecting system of claim 5, wherein the reservoir includes a first portion defined by internal volume of the sump and the at least one porous material and a second portion defined by the fluid impermeable layer and the at least one porous material; and
wherein the fluid path formed by the at least one porous material is between the first opening and the first portion of the reservoir.

7. The urine collecting system of claim 5, wherein the sump includes a bulbous portion extending from the outlets towards the proximal end, and wherein the bulbous portion at least partially defines the second section of reservoir.

8. The urine collecting system of claim 2, wherein the at least one porous material extends from the proximal end to and into the distal end.

9. The urine collecting system of claim 2, wherein the second opening is offset from a center of the base portion.

10. The urine collecting system of claim 2, wherein the base portion includes one or more rounded corners.

11. The urine collecting system of claim 2, wherein the base portion includes a bottom surface configured to abut one or more regions about the penis of the user, the base surface including an adhesive.

12. The urine collecting system of claim 11, wherein the first attachment mechanism is distinct from the bottom surface.

13. The urine collecting system of claim 2, wherein the primary attachment portion is a region of the base portion configured to be attached to a pubic mound of the user and the secondary attachment portion is a region of the base portion configured to be attached to at least regions of the user on lateral sides of a penis and a scrotum of the user.

14. The urine collecting system of claim 2, wherein the base portion exhibit a maximum length measured perpendicular to the maximum width, the maximum length is greater than the maximum width.

15. The urine collecting system of claim 2, wherein the base portion defines one or more slits extending from an outer periphery of the base portion towards the second opening.

16. The urine collecting system of claim 2, wherein the base portion is flexible.

17. The urine collecting system of claim 2, wherein a width of the base portion tapers from the maximum width to the minimum width.

18. The urine collecting system of claim 2, wherein an outer periphery of at least a portion of the secondary attachment portion exhibits a shape that corresponds to a shape of the second opening.

19. The urine collecting system of claim 2, wherein the first attachment mechanism defines one or more first vents extending therethrough and a second attachment mechanism of the body defines one or more second vents extending therethrough.

20. A urine collecting system, comprising:
a urine collecting assembly including a body, the body including a proximal end and a at least partially closed distal end, the body defining a first opening at or near the proximal end, the body including:
a sheath extending from or near the proximal end to or near the at least partially closed distal end, the sheath including at least one fluid impermeable layer, the sheath at least partially defining a reservoir; and
a sump at the at least partially closed distal end of the body, the sump defining an interior volume and including an outlet extending from the interior volume, the sump is distinct and separate from the at least one fluid impermeable layer, the sump attached to the at least one fluid impermeable layer; and
at least one porous material extending from or near the outlet to the proximal end, the at least one porous material adjacent to the fluid impermeable layer;
a stabilization accessory defining a second opening therethrough, the stabilization accessory comprising:
a flexible base portion including a bottom surface configured to abut one or more regions about the penis of the user, the base surface including an adhesive, the base portion including a primary attachment portion configured to be attached to a pubic mound of a user and a secondary attachment portion configured to be attached to at least regions of the user on lateral sides of a penis and a scrotum of the user, the base portion exhibits a maximum width measured perpendicular to a longitudinal axis of the body, and wherein the primary attachment portion includes at least a portion of the maximum width, wherein the secondary attachment portion includes an apex forming the minimum width of the stabilization accessory and the minimum width is spaced from and on an opposite side of the second opening from at least a portion of the primary portion including the maximum width, the base portion exhibiting one or more rounded corners; and
a first attachment mechanism distinct from the bottom surface and configured to attach the stabilization accessory to the body; and
wherein the reservoir is defined by at least the outlet, the at least one porous material, and the fluid impermeable layer, such that the porous material forms a fluid path between the first opening and the reservoir.

21. A urine collecting system, comprising:
a urine collecting assembly including a body, the body including a proximal end and a distal end, the body defining a first opening at or near the proximal end, the body including:
a sheath extending from or near the proximal end to or near the distal end, the sheath including at least one fluid impermeable layer, the sheath at least partially defining a reservoir, a portion of the sheath at or near the distal end defines a hole;
a top porous layer that at least partially covers the hole defined by the sheath;
a sump at or near the at least partially closed distal end of the body, the sump defining an interior volume and including an outlet extending from the interior volume, the sump is distinct and separate from the at least one fluid impermeable layer, the sump attached to the at least one fluid impermeable layer; and
at least one porous material extending from or near the outlet to the proximal end, the at least one porous material adjacent to the fluid impermeable layer;
a stabilization accessory attached to the body and defining a second opening therethrough that is aligned with the first opening, the stabilization accessory comprising:
a base portion including a bottom surface configured to abut one or more regions about the penis of the user, the base surface including an adhesive, the base portion including a primary attachment portion and a secondary attachment portion, the base portion exhibits a maximum width measured perpendicular to a longitudinal axis of the body, and wherein the primary attachment portion includes at least a portion of the maximum width, wherein the secondary attachment portion includes an apex forming the minimum width of the stabilization accessory and the minimum width is spaced from and on an opposite side of the second opening from at least a portion of the primary portion including the maximum width; and
wherein the reservoir is defined by at least the outlet, the at least one porous material, and the fluid impermeable layer, such that the porous material forms a fluid path between the first opening and the reservoir.
